(19) United States
(12) Patent Application Publication
(10) Pub. No.: US 2024/0255485 A1
(43) Pub. Date: Aug. 1, 2024

(54) WATER MONITORING AND CONTROL SYSTEM INCORPORATING REAL-TIME RAINFALL AND WATER QUALITY DATA IN PARALLEL

(71) Applicant: Flowlink Environmental Inc., Coquitlam (CA)

(72) Inventors: Elena RANYUK, Coquitlam (CA); Guillaume LANGLOIS, Coquitlam (CA); Thomas E G HAYES, Coquitlam (CA)

(73) Assignee: Flowlink Environmental Inc., Coquitlam, BC (CA)

(21) Appl. No.: 18/162,267

(22) Filed: Jan. 31, 2023

(51) Int. Cl. G01N 33/18; C02F 1/00; G01W 1/14

(52) U.S. Cl. CPC G01N 33/18; C02F 1/008; G01W 1/14; C02F 2209/006; C02F 2209/008; C02F 2209/02; C02F 2209/11; G01W 2203/00

(57) ABSTRACT

There is provided a real-time water monitoring and control system. The system includes sensors via which water quality, flow and system data is measured. The system includes a rain gauge or a weather application programming interface integration to obtain rainfall data in real-time. The system includes a processor configured to determine in real time whether the rainfall data satisfies a set of one or more predetermined rainfall conditions and in parallel or sequentially, in any order, configured to determine in real time whether one or more of the water quality, flow and system data satisfies a set of one or more of predetermined conditions. If so, the processor is configured to send a signal to activate one or more of a notification, a water flow device and an automated sampling device. The processor sends a signal to deactivate all triggered actions when any of said predetermined conditions is no longer satisfied.

### BACKGROUND OF THE INVENTION

### Field of the Invention

There is provided a water monitoring and control system. In particular, there is provided a water monitoring and control system incorporating real-time rainfall and water quality data in parallel.

### Description of the Related Art

United States Patent Application Publication No. 2010/0332149 A1 to Scholpp discloses a remote monitoring system for monitoring the operation of a fluid treatment system and/or the qualities, characteristics, properties, etc., of the fluid being processed or treated by the fluid treatment system. The system for measuring fluid quality and/or equipment operation in a fluid treatment system includes a remote computer that may be associated with a database that accesses data transmitted from the fluid treatment system with the data collected, acquired, etc., from one or more sensors placed in the fluid treatment system. The data may be analyzed or manipulated by a local computer and/or the remote computer, which may be used to generate an analysis result or analysis report. Such results or reports, along with any other information or data including historical or expected information, may be sent or communicated to a remote viewing device for viewing by a user. Methods are further provided for the operation of the remote monitoring system of the present invention.

U.S. Pat. No. 11,392,841 to Alsubai et al. discloses a system to monitor and process water-related data. The system includes a plurality of water-related sensors which each include an environmental characteristic detection element, a power source, and a communication device to transmit data associated with water-related data at a site. A water impact data store may contain electronic records associated with prior water-related events at other sites along with water-related sensor location data for those sites, and a third-party information interface may receive third-party information. An enterprise analytics platform may automatically analyze the electronic records in the water impact data store to create a predictive analytics algorithm. The data associated with potential water-related data at the site and the third-party information may then be automatically analyzed, in substantially real-time, using the predictive analytics algorithm, and a result of the analysis may then be transmitted (e.g., to a party associated with the site and/or an onsite water shut-off valve).

United States Patent Application Publication No. 2019/0257068A1 to Conway discloses a system and method for containing environmental spills at a zone, for example at a port or rail facility, in order to prevent run-off water from contaminating the environment. The system and method relates to a water parameter sensor system which is compared to stored contaminants to establish the nature of the contaminant. The system and method further relates to the comparison of the identified contaminant to a manifest database to be able to establish where a spill or leakage may be found. The system comprises a rain sensor operably coupled to the controller and wherein, in use, the rain sensor is adapted to determine a rain event. The system includes a controller adapted to, upon detection of a rain event and detection of a contaminant, close the discharge valve. In one embodiment the controller is adapted to, upon detection of a rain event, open the discharge valve, and actuate a sampling sequence.

U.S. Pat. No. 6,077,423 to Roy et al. discloses a method and system for treating storm water runoff containing impurities includes collecting the runoff in a basin and allowing the runoff to settle in the basin for a predetermined time before allowing the filtration step to be initiated. The time delay is controlled by a controller sensitive to rain fall, turbidity, or other variables selected by the user. During the filtration step a separator member is positioned floatingly between a filter element and impurities floating on the surface of the unfiltered water.

### BRIEF SUMMARY OF INVENTION

There is provided, and it is an object to provide, an improved water monitoring and control system and process disclosed herein.

There is accordingly provided a real-time water monitoring and control system according to a first aspect. The system includes a plurality of sensors configured to measure properties of the water in real-time. The system includes a processor in communication with the sensors and rainfall data in real-time. The processor is configured to cause the water to discharge based on whether the water is below a first water quality threshold and the processor is configured to cause the water to discharge based on whether the water is below a second water quality threshold where a rainfall condition has been met.

There is further provided a real-time water monitoring and control system according to a second aspect. The system includes one or more sensors via which water quality, flow and system data is measured. The system includes one or more of rain gauge and a weather application programming interface (API) integration to obtain rainfall data in real-time. The system includes a processor configured to determine in real time whether the rainfall data satisfies a set of one or more predetermined rainfall conditions and in parallel or sequentially, in any order, configured to determine in real time whether one or more of the water quality, flow and system data satisfies a set of one or more of predetermined conditions. If so, the processor is configured to send a signal to activate one or more of a notification, a water flow device and an automated sampling device. The processor is configured to send a signal to deactivate all triggered actions when any of said predetermined conditions is no longer satisfied.

There is also provided a real-time water monitoring and control system according to a third aspect. The system includes one or more sensors configured to measure water quality, flow and system data in real-time. The system includes a rainfall acquisition device configured to obtain rainfall data in real-time. The system includes a processor configured to determine whether the rainfall data exceeds one or more predetermined thresholds. If the rainfall data exceeds one or more predetermined thresholds, the processor is configured to determine whether one or more of the water quality, flow and system data exceeds a first set of one or more predetermined thresholds. If so, the processor is configured to send a first signal to activate one or more of a first notification and a water flow device. If the rainfall data is below the one or more predetermined thresholds, the processor is configured to determine whether one or more of the water quality, flow and system data exceeds a second set of one or more predetermined thresholds. If so, the processor is configured to send a second signal to activate one or more of a second notification and said water flow device.

There is additionally provided a real-time water monitoring and control process according to a first aspect. The process includes obtaining rainfall data in real-time via one or more of a rain gauge and a weather application programming interface (API). The process includes measuring water quality, flow and system data in real-time via one or more sensors. The process includes determining in real time via a processor whether the rainfall data satisfies a set of one or more predetermined rainfall conditions. The process includes in parallel or sequentially, in any order, determining in real time whether one or more of the water quality, flow and system data satisfies a set of one or more of predetermined conditions. If so, the process includes sending a signal to activate one or more of a notification, a water flow device and an automated sampling device. The process includes sending a signal to deactivate all triggered actions when any said predetermined condition is no longer satisfied.

There is further provided a real-time water monitoring and control process according to a second aspect. The process includes obtaining rainfall data in real-time and measuring water quality, flow and system data in real-time via one or more sensors. The process includes determining via a processor whether the rainfall data exceeds one or more predetermined thresholds. If the rainfall data exceeds one or more predetermined thresholds, the process includes determining whether one or more of the water quality, flow and system data exceeds a first set of one or more predetermined thresholds. If so, the process includes sending a first signal to activate one or more of a first notification and a water flow device. If the rainfall data is below the one or more predetermined thresholds, the process includes determining whether one or more of the water quality, flow and system data exceeds a second set of one or more predetermined thresholds. If so, the process includes sending a second signal to activate one or more of a second notification and said water flow device.

There is yet also provided a real-time water monitoring and control process according to a third aspect. The process includes continuously monitoring water at a construction site having wastewater. The process includes automatically sending a first signal to activate one or more control processes upon determining that one or more of water quality, flow and system data exceeds one or more predetermined value thresholds for greater than a first predetermined amount of time. The process includes automatically sending a second signal to deactivate the one or more control processes upon determining the one or more of water quality, flow and system data is below the one or more predetermined value thresholds for greater than a second predetermined amount of time.

There is yet additionally provided a method of automatically monitoring and controlling water. The method includes obtaining rainfall data in real-time. The method includes determining via a processor whether the rainfall data for a first predetermined amount of time exceeds a predetermined threshold and if so recording that a new significant rainfall event has occurred and determining that a rainfall condition has been met. If the processor determines that the rainfall data for the first predetermined amount of time does not exceed the predetermined threshold, the method includes determining via the processor whether a previous significant rainfall event was recorded and if no, determining that the rainfall condition has not been meet. If the processor determines that the rainfall data for the first predetermined amount of time does not exceed the predetermined threshold and determines that the previous significant rainfall event was recorded, the method includes determining via the processor whether the previous significant rainfall event was recorded at a time equal to or less than a second predetermined amount of time and if so, determining that the rainfall condition has been met. If the processor determines that the rainfall data for the first predetermined amount of time does not exceed the predetermined threshold, determines that the previous significant rainfall event was recorded, and determines that the previous significant rainfall event was recorded at a time greater than the second predetermined amount of time, then the method includes determining via the processor that the rainfall condition has not been met. The method includes determining whether water is to be discharged based on different water quality criteria depending on whether the rainfall condition has been met.

There is further provided a method of automatically monitoring and controlling water. The method includes providing at least one rule in a memory of a water monitoring and control system. The rule includes at least one parameter and when triggered, the method causes a signal to activate one or more of a notification, a water flow device and an automated sampling device of the water monitoring and control system. The method includes determining via a processor of the system whether the rule has been triggered. If the processor determines that the rule has been triggered, the method includes determining via the processor whether the at least one parameter meets a predetermined threshold for a predetermined deactivation duration and if yes, reporting that a parameter threshold condition is not met and causing the rule to no longer be triggered. If no, the method includes determining via the processor that the parameter threshold condition is met. If the processor determines that the rule has not been triggered, the method includes determining via the processor whether the at least one parameter exceeds the predetermined threshold for a predetermined activation duration and if so, the method includes determining via the processor that the parameter threshold condition is met and causing the rule to be triggered. If no, the method includes determining via the processor that the parameter threshold condition is not met.

It is emphasized that the invention relates to all combinations of the above features, even if these are recited in different claims.

Further aspects and example embodiments are illustrated in the accompanying drawings and/or described in the following description.

### BRIEF DESCRIPTION OF DRAWINGS

The accompanying drawings illustrate non-limiting example embodiments of the invention.

FIG. 1 is a schematic flow diagram of the processing of influent through a water treatment system so as to output an effluent, together with a water monitoring and control system according to one aspect, with the effluent monitoring and control system analyzing the effluent so outputted and based on the same directing the effluent to either be discharged or re-circulated back to the influent or any other collection point;

FIG. 2 is a schematic flow diagram similar to FIG. 1 without a water treatment system, where influent is analyzed by the water monitoring and control system of FIG. 1 and directed to either be discharged or re-circulated based on water quality thereof;

FIG. 3 is a schematic flow diagram of the water monitoring and control system of FIG. 1, with the water monitoring and control system including a remote unit comprising a controller, water quality sensors, flow sensors, a flow control device and optionally a rainfall gauge, and the water monitoring and control system including a remote computer system comprising a remote communication system operatively connected to the controller, a data acquisition system operatively connected to both the communication system and a weather application programming interface (API) for rainfall data, and a processor operatively connected to and which receives data from the data acquisition system, the processor also operatively connected to the remote communication system;

FIG. 4 is a schematic flow diagram showing the general overall operation and process of the water monitoring and control system of FIG. 3;

FIG. 5 is a schematic flow diagram showing the general evaluation by the processor of FIG. 3 of the conditions of the water monitoring and control system so as to determine whether rules have been triggered and corresponding actions need to be activated or deactivated;

FIG. 6 is a schematic flow diagram showing a process of determining whether a water quality, flow or system data condition of the water monitoring and control system of FIG. 4 is satisfied;

FIG. 7 is a schematic flow diagram showing a process of determining whether a rainfall condition of the water monitoring and control system of FIG. 4 is satisfied;

FIG. 8 is a schematic flow diagram showing a process of determining whether a parameter threshold condition of the water monitoring and control system of FIG. 4 is satisfied;

FIG. 9 is a schematic flow diagram of processing of rule actions for the processor of the water monitoring and control system of FIG. 4;

FIG. 10 is a non-limiting example of a user interface for creating, adjusting and/or customizing a rule for the rule engine of the water monitoring control system of FIG. 4; and

FIG. 11 is a non-limiting example of a dashboard interface for the water monitoring control system of FIG. 4.

### DESCRIPTION OF THE PREFERRED EMBODIMENTS

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive sense.

Referring to the drawings and first to FIG. 1, there is shown schematically a water treatment, monitoring and control assembly/process 30 which may be situated at a site where water discharged therefrom is monitored, such as a development or construction site 31 in one non-limiting embodiment. The construction site may comprise water of a highly variable quality, with a pH in the range of 4 to 12 and a turbidity level in the range of 0 to 500 NTU (or greater) in one non-limiting embodiment. Water quality may refer to the physical, chemical and/or biological properties of the effluent.

Influent 32 may have properties that are outside of parameters/specifications for what is acceptable for discharge into public bodies of water according to given local, provincial/state or Federal bylaws or regulations. In this case the jurisdiction may require that no water discharged into a drainage system be outside of a given water specifications or exceed one or more water quality thresholds. In one non-limiting example, the local jurisdiction may require that no water be discharged having a pH value outside of the range of 6.5 to 8.0 nor that any water be discharged into a drainage system with a turbidity level greater than 25 nephelometric turbidity units (NTU), except during and for 24 hours after a significant rainfall event (e.g. >25 mm per 24 hour period) in which case the water to be discharged may not exceed 100 NTU. These thresholds may vary depending on a given jurisdiction and its associated regulatory requirements.

As seen in FIG. 1, process 30 comprises directing influent 32 into a water treatment system 34 downstream of the influent, in this example via a conduit 36. The water treatment system may subject the influent to various chemical, biological and/or physical treatments to try to ensure that treated water or effluent 38 outputted from water treatment system 34 via conduit 40, is within water specifications of a given jurisdiction such as that set out above. The effluent may be referred to as a wastewater. Water treatment systems per se, including their various parts and functionings, are well known to those skilled in the art and water treatment system 34 accordingly will not be discussed in further detail.

Referring to FIG. 1, process 30 includes a real-time water monitoring and control system 42 downstream of water treatment system 34, in this example downstream of effluent 38 and in fluid communication therewith in this example via conduit 44. The water monitoring and control system is configured to determine one or more water qualities of the effluent and if the one or more water qualities so determined are within the local jurisdiction's water specifications, the water monitoring and control system enables passage of the effluent to a discharge location 46 via conduit 48 in this example. If the one or more water qualities of effluent 38 so determined are not within water specifications as determined by real-time water monitoring and control system 42, the water monitoring and control system is configured to inhibit discharge of the effluent and re-circulate the effluent as shown by box 49 to water treatment system 34 in this case via conduit 50. The water monitoring and control system may thus be called an effluent monitoring and control system in this configuration.

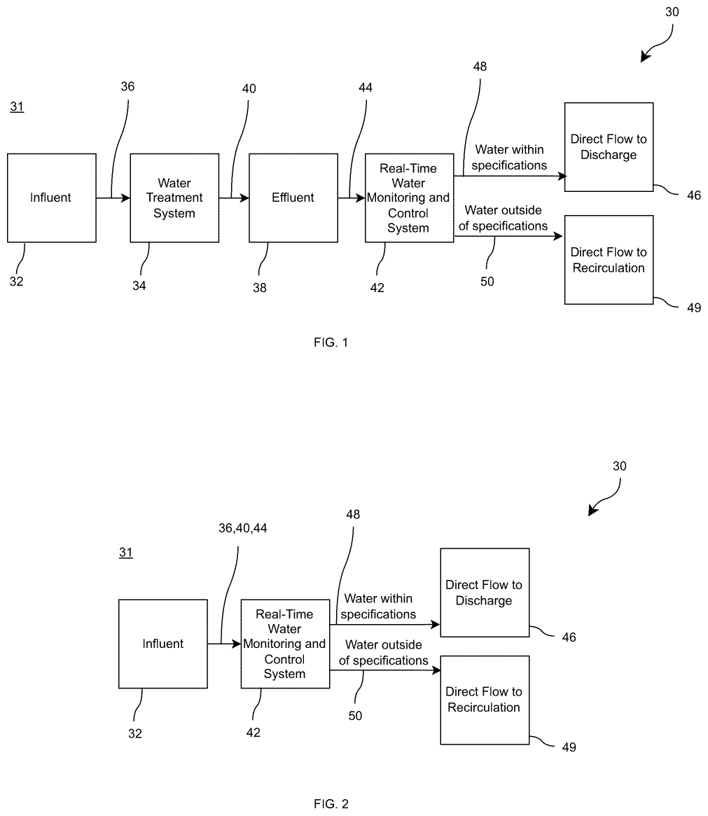

Alternatively and as shown in FIG. 2, a water treatment system is not strictly required for use in association with real-time water monitoring and control system 42. In this case the water monitoring and control system is positioned downstream of influent 32 and recirculates water which is not within water specifications so determined by the water monitoring and control system directing it to the influent or another collection point.

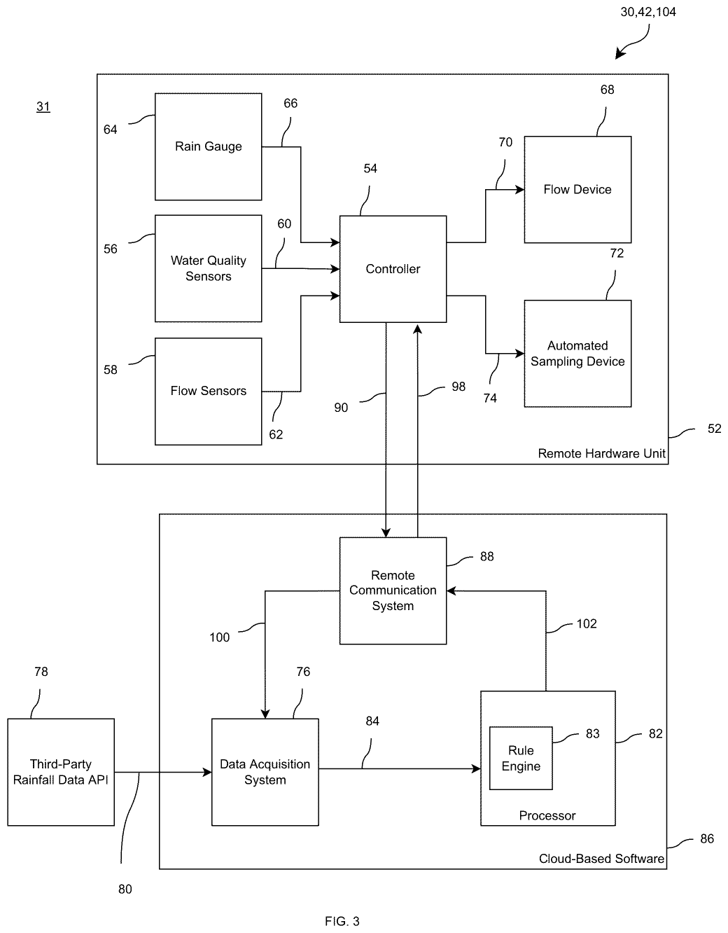

Referring to FIG. 3, the various components of real-time water monitoring and control system 42 will now be described in further detail.

Water monitoring and control system 42 includes an onsite unit 52 configured to be situated upon site 31. The onsite unit may be referred to as a remote unit. Onsite unit 52 includes various hardware including a controller 54. The onsite unit includes a plurality of sensors including water quality sensors 56 and one or more flow sensors 58. The water quality sensors are configured to measure one or more water qualities of effluent 38 seen in FIG. 1 relevant to the water specifications of the local jurisdiction, including in one non-limiting embodiment temperature and pH and turbidity levels. The one or more flow sensors are configured to measure/determine the rate of flow of effluent passing into real-time water monitoring and control system 42 and/or being recirculated via or passing through the water monitoring and control system. Each of sensors 56 and 58 is configured to output data indicative of one or more water qualities or flow of the effluent via one or more signals 60 and 62 in real-time. Controller 54 operatively connects to the sensors and receives said signals therefrom in this non-limiting embodiment.

Still referring to FIG. 3, real-time water monitoring and control system 42 in this example includes a rainfall sensor or gauge 64, though this is not strictly required. The rainfall gauge outputs in real time rainfall information or data in this example via one or more signals 66. Controller 54 operatively connects to rainfall gauge 64 and receives said signals therefrom in this non-limiting embodiment. The rainfall gauge in this example is a part of onsite unit 52, though this is not strictly required. Rainfall gauge 64 is situated on or near site 31 and may, alternatively, comprise a third party off-the-shelf component that operatively connects to the onsite unit.

Onsite unit 52 includes a flow device 68. The flow device may comprise a valve which selectively enables or inhibits discharge of effluent. In addition or alternatively, flow device 68 may comprise a pump, with actuation thereof causing effluent to discharge and/or recirculate. Controller 54 operatively connects to and is configured to selectively actuate the flow device as shown by arrow 70. Flow device 68 has a first or open position in this example configured to enable effluent 38 to pass through real-time water monitoring and control system 42 through to discharge location 46 seen in FIG. 2. The flow device is movable via the controller from the open position to a second or closed position. Flow device 68 is configured in the closed position thereof in this example to redirect/recirculate effluent towards influent 32 and/or water treatment system 34. These configurations are not strictly required and opening the flow device may promote recirculation and closing the flow device may promote discharge in other embodiments, for example.

Onsite unit 52 includes an automated sampling device 72. Controller 54 operatively connects to and is configured to selectively actuate the automated sampling device as shown by arrow 74. Rain gauges, water quality sensors, flow sensors, controllers, flow devices and automated sampling devices per se, including their various parts and functionings, are known to those skilled in the art and rain gauge 64, sensors 56 and 58, controller 54, flow device 68 and automated sampling device 72 will accordingly not be described in further detail.

Still referring to FIG. 3, real-time water monitoring and control system 42 in this example includes a data acquisition system 76. The data acquisition system in this non-limiting embodiment operatively connects to a weather application programming interface (API) 78 for rainfall data as seen by arrow 80. This may be a third party weather API, with data acquisition system 76 being configured to automatically and continually query the weather API for weather data tied to specific parameters, in this non-limiting example rainfall data that is location and time specific. The location specified is that of or adjacent to site 31 in this example, with the data related thereto thus being acquired in real-time and continually. Rain gauge 64 and/or weather API 78 may be referred to individually or collectively as a rainfall acquisition device.

Water monitoring and control system 42 includes a processor 82 and a rule engine 83 with which the processor operates and/or which is a part of the processor. The rule engine may be referred to as a software system or rule engine software. Processor 82 may include intelligent software and/or include artificial intelligence (AI) features. The terms processor and rule engine may be used interchangeably herein and in the description that follows. Processor 82 and rule engine 83 drive action (hardware) of real-time water monitoring and control system 42 for specific uses as will be described further below. The processor and rule engine operatively connect to and receive data from data acquisition system 76, as shown by arrow 84. In this non-limiting embodiment, the data acquisition system, processor 82 and rule engine 83 are situated at a remote location spaced-apart from site 31, in this example being part of a remote server 86 (e.g. cloud computing). However, this is not strictly required and data acquisition system 76, processor 82 and rule engine 83 may be part of onsite unit 52 and/or controller 54 thereof in other embodiments, for example.

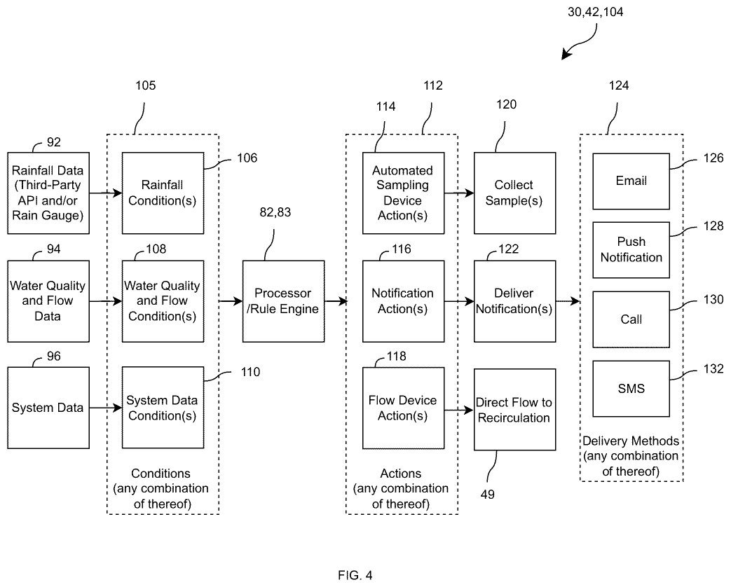

Water monitoring and control system 42 includes a communication system, in this example a remote communication system 88 operatively connected to and in communication with controller 54. The communication system is configured to receive signals and/or data from the controller, as shown by arrow 90, such as rainfall data 92, water quality and flow data 94 and system data 96 seen in FIG. 4. This data is obtained in real time from sensors 56 and 58 of onsite unit 52 seen in FIG. 3 as well as, for example, rain gauge 64. Still referring to FIG. 3, communication system 88 is configured to transmit signals and/or data to the controller, as shown by arrow 98, such as signals comprising instructions to the controller for selectively actuating flow device 68, automated sampling device 72 or the like. The above set out communication may occur via a transceiver or other functionally equivalent device or means as would be known to someone skilled in the art.

Still referring to FIG. 3, communication system 88 operatively connects to and sends data from controller 54 to data acquisition system 76, as shown by arrow 100. The communication system operatively connects to and receives signals or instructions from processor 82, as shown by arrow 102.

Referring to FIG. 4, a water treatment and control process 104 for real-time water monitoring and control system 42 is generally shown. The process comprises measuring or obtaining rainfall data 92 (via weather API 78 and/or rain gauge 64), water quality and flow data 94 (e.g. effluent pH, temperature, turbidity level, conductivity, flow rate etc.) and system data 96 (e.g. valve open/closed, operation state of sensors/controller/sampling-device etc.). Data is received by processor 82 seen in FIG. 3, in this example via controller 54, communication system 88 and data acquisition system 76, to determine whether a plurality of conditions 105 seen in FIG. 4 have been met including: rainfall condition(s) 106, water quality and flow condition(s) 108, system data condition(s) 110 and/or any combination thereof. The following is non-limiting example of a rainfall condition: has 25 mm of rain fallen within the last 24 hours? The following is a non-limiting example of a water quality and flow condition: has the pH of the effluent been greater than 9 for 5 or more minutes while flow was detected?

System data conditions 110 may include unit state (e.g. active, idle, maintenance, offline) for respective components of real-time water monitoring and control system 42 such as onsite unit 52 or flow device 68 (e.g. a valve and/or pump) seen in FIG. 3. The system data may include information on whether the flow device thereof is an open or closed position for example. Another non-limiting example of a system condition comprises: is sensor data missing and/or not communicating? For example, there may be an issue with one of sensors 56 and 58 seen in FIG. 3 detected by periodic pinging of the sensors with no responsive to the same. A rule in this case may be triggered if there is no data transmission for a predetermined amount of time (e.g. 15 minutes), with the rule including a notification action to notify one or more designated persons. This may in turn trigger a response from a technical support team to evaluate real-time water monitoring and control system 42 and/or replace the faulty sensor.

Referring back to FIG. 4, rule engine 83 includes a plurality of customizable rules that may be created on a per-project basis. The rule engine may thus be said to comprise a general algorithm that enables the operator, regulatory body and/or end-user to readily tailor the same to any municipal requirements. An unlimited number of conditions 105 and actions 112 can be associated with a rule. Multiple types of rules are possible for the conditions and actions. A rule itself does not need any knowledge about the type of conditions 105 and actions 112—that is any combination of conditions is acceptable and rule engine 83 may be configured to be process-agnostic and/or site-agnostic.

Non-limiting examples of actions for real-time water monitoring and control system 42 include: automated sampling device action(s) 114, notification action(s) 116, flow device action(s) 118 and/or any combination thereof.

When the rule is processed, all associated conditions 106, 108 and/or 110 are evaluated. If all conditions are satisfied (i.e.: return true), the rule status changes to triggered and all associated actions 114, 116 and/or 118 will be started.

When a rule associated with automated sampling device action(s) 112 is triggered, automated sampling device 72 seen in FIG. 3 is actuated to collect sample(s) of effluent as seen by box 120 in FIG. 4.

Referring back to FIG. 4, when a rule associated with notification action(s) 116 is triggered, processor 82 is configured to send/deliver one or more signals in the form of notification(s) 122. The notifications may comprise one or more delivery methods 124, including but not limited to: email 126, one or more push notifications 128 on an application or mobile device, one or more telephone calls 130 leaving an automated notification/message or otherwise, one or more text messages or short message service (SMS) 132, and/or any combination thereof. Notifications and data-sharing may be configured to occur in real-time. System 42 includes an ability to customize the person or persons to whom the notification(s) should be sent, with more common notifications being sent to a more operations specific person and more urgent notifications being also sent to high level managers, owners or the like, for example. Notification actions may also be configured to be sent to various third parties and/or regulatory bodies as required/desired.

Still referring to FIG. 4, when a rule associated with flow device action(s) 118 is triggered, flow device 68 seen in FIG. 3 is actuated from the open position thereof to the closed position to direct flow of effluent to recirculation 49.

Referring back to FIG. 4, real-time water monitoring and control system 42, including rule engine 83 thereof as herein described, enables a flexibility and customization to its rule-making that may enable the detection of unlimited site situations of interest. A non-limiting example of a rule to this end comprises:
a. is the pH of the effluent greater than a first predetermined threshold (e.g. pH>9) for a first predetermined period of time (e.g. 5 minutes) and/or
b. is the turbidity level of the effluent greater than a second predetermined threshold (e.g. 25 NTU) for a second predetermined period of time (e.g. 15 minutes) and/or
c. is there no significant rainfall event (defined as no rainfall exceeding a third predetermined threshold such as 25 mm, for a third predetermined period of time such as 24 hours)?
d. if all of the above conditions are met, then the rule is triggered and as part of the actions associated with the rule so triggered, processor 82:
   i. initiates a flow device action 118 to close flow device 68 seen in FIG. 3 so as to direct effluent flow to recirculate 49, thereby inhibiting discharge of the effluent, and/or
   ii. initiates a notification action 116 seen in FIG. 4 in the form of a push notification 128 to one or more designated end users e.g. a site manager or regulatory official, indicating that effluent is recirculating and not being discharged.

Rules comprising predetermined thresholds and conditions along the above or other lines, may thus be customized to given local, provincial/state or Federal bylaws or regulations which may vary widely. On a subsequent rule evaluation, if any of the conditions is not satisfied, all associated actions will be stopped and the rule will no longer be triggered. This occurs based on parameter threshold conditions or activation/deactivation thresholds as will be discussed further below. When rules cease to be triggered, a log or record is created and saved into memory of real-time water monitoring and control system 42 for tracking purpose.

The evaluation of conditions, parameter threshold conditions for activation or deactivation of rules and the processing of rule actions will now be described in further detail.

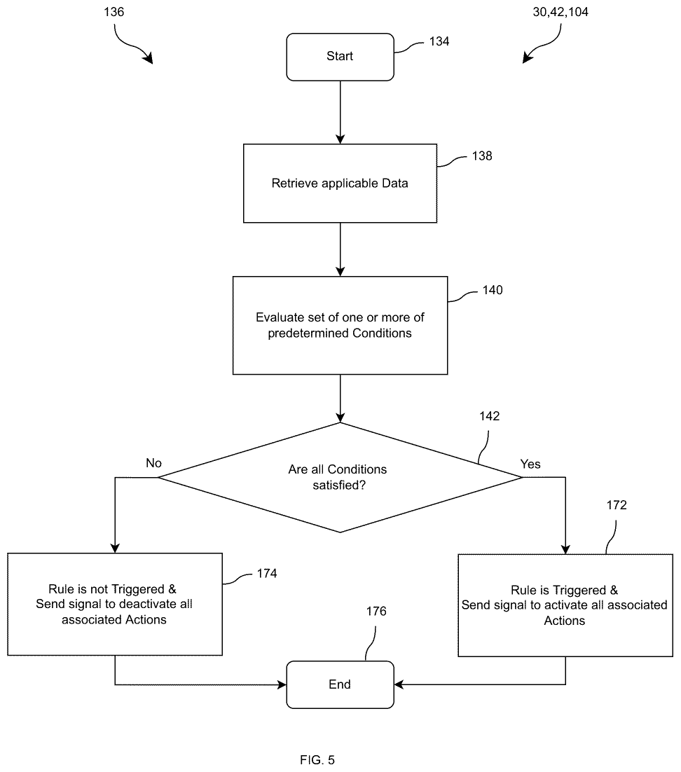

To determine whether a condition is satisfied and referring to FIG. 5, the processor starts (box 134) a condition evaluation process 136. As part of the process, the applicable data (e.g. rainfall data, water quality/flow data, system data etc.) for the condition to be evaluated is retrieved as shown by box 138. In a non-limiting embodiment for system data this may comprise determining the state data for each sensor of real-time water monitoring and control system 42. Condition evaluation process 136 includes next evaluating this data against or with respect to a set of one or more predetermined conditions as shown by box 140. For the system data example, the predetermined conditions may comprise each of the sensors must have a state of operational with power for a predetermined time period (e.g. 15 minutes). The processor next determines whether all of the conditions of a given rule are satisfied as shown by box 142.

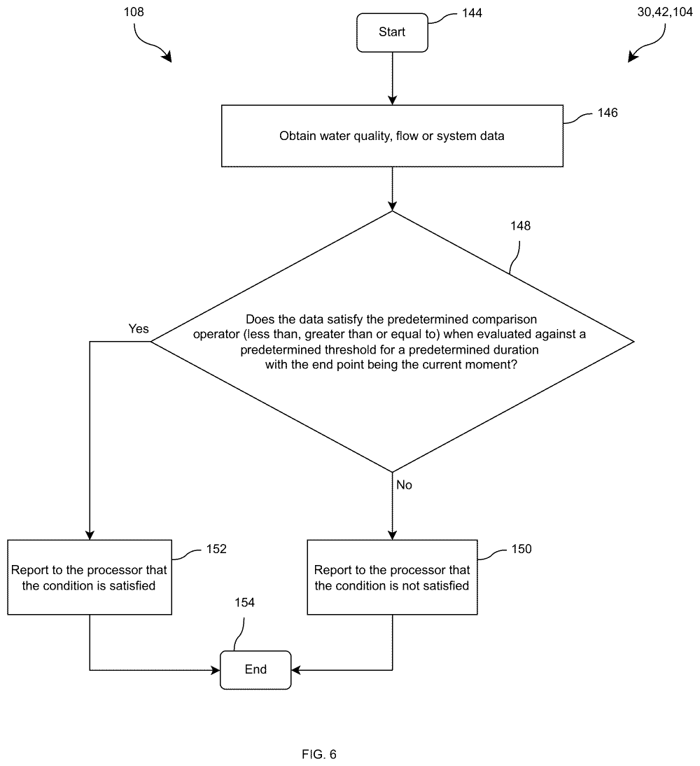

FIG. 6 shows a non-limiting embodiment for determining whether a water quality, flow and/or system data condition is satisfied. The processor starts (box 144) the process by first obtaining water quality, flow and/or system data as shown by box 146. As seen by box 148, the processor next determines whether the data satisfies the predetermined comparison operator (i.e. less than, greater than, or equal to) when evaluated against a predetermined threshold for a predetermined duration with the end point being the current moment. For example: i) a water quality condition may be that the pH of the effluent must not be outside of the range of 6.5 and 8.0 for a period greater than 15 minutes, ii) the predetermined threshold may be a pH of equal to or greater 6.5 and equal to or less than 8.0 and iii) the data may indicate that the effluent has a pH of 5 during this time period, with the water quality condition thus not being satisfied. Another condition may be that the turbidity of the effluent must not be for a period greater than 30 minutes: greater than 25 NTU where there is no significant rain event (which for example may be defined/customized based on a rainfall amount over a rolling period of time) and less than 100 NTU when there is a significant rain event. Another condition may be that the state of the real-time water monitoring and control system 42 must not be offline for a period greater than 5 minutes.

Referring back to FIG. 6, if the data is determined to not satisfy the predetermined comparison operator when evaluated against a predetermined threshold for a predetermined duration with the end point being the current moment, the processor determines or the rule engine reports to the processor that the given condition which is being analyzed is not satisfied as shown by box 150. If the data is determined to satisfy the predetermined comparison operator when evaluated against a predetermined threshold for a predetermined duration with the end point being the current moment, the processor determines or the rule engine reports to the processor that the given condition which is being analyzed is satisfied as shown by box 152.

The process of determining whether the given water quality, flow and/or system data condition is satisfied thereafter reaches its end 154 and may be re-run or polled at predetermined frequencies according to customizable system or rule settings. Water monitoring and control system 42 may be configured to poll sensors 56 and 58 seen in FIG. 3 at a predetermined frequency to continuously monitor/determine whether the water quality, flow and/or system data conditions are satisfied on a rolling window basis. In one non-limiting embodiment water quality may be tested every 30 seconds or every minute at most, with water monitoring and control thus effectively occurring in real-time.

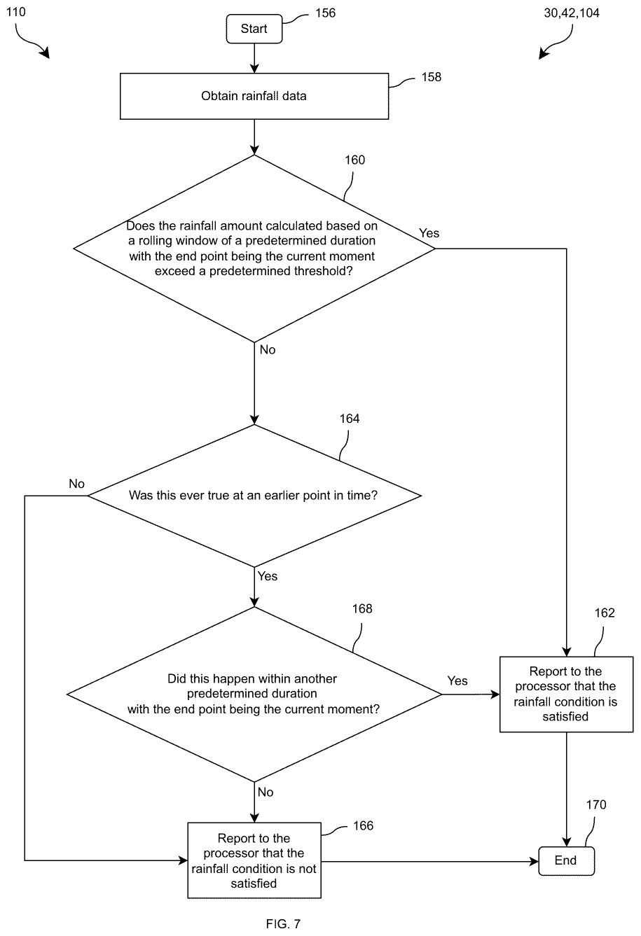

FIG. 7 shows a non-limiting embodiment for determining whether a rainfall condition is satisfied for real-time water monitoring and control system 42 as herein described. The processor starts (box 156) the process by first obtaining rainfall data as shown by box 158. This may be via rain gauge 64 and/or weather API 78 seen in FIG. 3. Referring back to FIG. 7 and as seen by box 160, the processor next calculates a rainfall amount based on a rolling window for a first predetermined duration or time period with the end point being the current moment and determines whether this exceeds a predetermined threshold. For example, the first predetermined period of time could be 12 hours, the predetermined threshold could be 25 mm, and the rainfall amount calculated for the last 12 hours could be 30 mm.

If the rainfall amount calculated in box 160 exceeds the predetermined threshold, then the processor determines or the rule engine reports to the processor that the rainfall condition is satisfied as shown by box 162. This may be used to determine that a significant rainfall event has or is occurring and that effluent should be analyzed/evaluated against water quality criteria reflecting the same.

If the rainfall amount calculated in box 160 does not exceed the predetermined threshold e.g. equals to 10 mm within the last 12 hours, in this case the processor is configured to query whether the rainfall amount over the first predetermined duration ever previously exceeded the predetermined threshold at an earlier point in time as shown by box 164. If no, then the processor determines or the rule engine reports to the processor that the rainfall condition is not satisfied as shown by box 166. This may be used to determine that no significant rainfall event has occurred and that effluent should be analyzed/evaluated against water quality criteria reflecting the same.

If the rainfall amount over the first predetermined duration did exceed the predetermined threshold at an earlier point of time, e.g. rainfall amount over 12 hour window did exceed 25 mm at some point in the past but not in the last 12 hours, then the processor next queries whether this occurred within a second predetermined duration or period of time with the end point being the current moment as shown by box 168. For example, the second predetermined period of time may be the last 48 hours based on a rolling window and it may have rained >25 mm during a 12-hour period at some point within the said 48 hours period. If such an event did occur within the second predetermined period of time per box 168, in this case the processor determines or the rule engine reports to the processor that the rainfall condition is satisfied as shown by box 162. This may be used to determine that a significant rainfall event has occurred and that effluent should be analyzed/evaluated against water quality criteria reflecting the same.

If no such event occurred within the second predetermined period of time per box 168, in this case the processor determines or the rule engine reports to the processor that the rainfall condition is not satisfied as shown by box 166. This may be used to determine that a significant rainfall event has not occurred and that effluent should be analyzed/evaluated against water quality criteria reflecting the same.

The process of determining whether the rainfall condition thereafter reaches its end 170 and may be re-run or polled at predetermined frequencies according to customizable system or rule settings. In one non-limiting embodiment the rainfall condition may be determined every 30 seconds or up to at least 60 seconds at the most, with real-time water monitoring and control system 42 thus effectively taking into account rainfall data in real-time. As a further non-limiting alternative, rainfall data may be obtained every hour with a rolling sum within a predetermined window such as the past 24 hours being calculated therefrom.

System 42 is thus configured to monitor rainfall in real-time and take action in response thereto. Thus, the system does not need to wait a prolonged period of time to determine whether a significant rainfall event has occurred or has been met. System 42 with its real-time polling is configured to determine when a significant rainfall event has occurred in a time effective/efficient manner, thereby maximizing the site's ability to discharge effluent at a more relaxed threshold and thus maximizing the site's ability to discharge effluent overall on the one hand, while also complying with regulatory thresholds on the other hand.

System 42 is therefore customizable to local water quality criteria, and analyses rainfall information so as to determine how this influences water quality criteria of effluent in real-time, thereby maximizing effluent discharge time in a manner that complies with local regulations. For example, a municipality located in or adjacent to mountains may be subject to more rain (e.g. clouds may be caught by the mountains and/or subject cooler mountain air, promoting increased precipitation thereby) and adopt different water quality discharge criteria as a result thereof, compared to a municipality that is spaced from mountains and which may be subject to less rain and more sunshine. System 42 as herein described with its rule engine flexibility, is configured to specifically adapt to and accommodate such and other varied conditions and/or regulatory requirements.

Referring back to FIG. 5, if all of the conditions are satisfied as shown by box 142, in this case the processor or rule engine thereof determine that the rule is triggered as shown by box 172 and the processor sends one or more signals to activate all association actions 112 seen in FIG. 4. Thus, if a given rule requires conditions A+B+C+D+E and they are all satisfied, then the given rule is triggered and associated actions of the rule will begin: e.g. if real-time water monitoring and control system 42 determines that A) the pH of the effluent is within the range of 6 to 8.5 for B) greater than 5 minutes, C) turbidity is less than 25 NTU for D) greater than 10 minutes and E) there is no significant rain event (e.g. less than 25 mm in the past 24 hours), then the rule may be constructed/configured to actuate flow device 68 seen in FIG. 3 to discharge effluent and send a notification to one or more designated persons of the same.

Another non-limiting example of a rule may comprise: is the pH of the effluent below 6.5 for equal to or greater than 5 minutes or above 8.0 for equal to or greater than 5 minutes? If so, the rule is triggered and the associated action is to automatically actuate the flow device to inhibit discharge of effluent and direct the flow of the effluent to recirculation instead and to notify one or more designated persons of the same.

Referring to FIG. 4, a further non-limiting example of a rule may comprise: is the turbidity of the effluent greater than 25 NTU for a period equal to or greater than 60 seconds and real-time water monitoring and control system 42 determines that rainfall condition 106 (i.e. no significant rainfall event) has not been meet? If so the rule is triggered and the associated action is to automatically actuate flow device 68 to inhibit discharge of effluent and direct the flow of the effluent to recirculation instead and to notify one or more designated persons of the same.

When rules are triggered, real-time water monitoring and control system 42 is configured to create a log or record of the same for tracking purpose. The real-time water monitoring and control system as herein described is configured to provide full transparency and auditability of the data thereof, which may be desirable for third parties such as regulatory bodies. On a subsequent rule evaluation which may occur automatically according to a predetermined/customization frequency, if any of the conditions is not satisfied, all associated actions will be stopped. This may be referred to as a deactivation rule.

Referring back to FIG. 5, if the processor or rule engine thereof determine that not all of the conditions are satisfied, in this case as shown by box 174 the processor or rule engine thereof determine that the rule is not triggered and the processor sends one or more signals to deactivate all associated actions. Therefore, if only some conditions are satisfied, then the rule is not triggered. Thus, if the rule requires conditions A+B+C+D+E in the example above, and real-time water monitoring and control system 42 determines that A) the pH of the effluent is within the range of 6 to 8.5 for B) greater than 5 minutes, C) turbidity is at times less than 25 NTU but not for D) greater than 10 minutes and E) there is no significant rain event (less than 25 mm in the past 24 hours), then the rule is not triggered because the turbidity of the effluent is too high, the flow device causes effluent to recirculate and a notification may be sent to one or more designated persons of the same on a predetermined periodic basis.

Condition evaluation process 136 thereafter ends 176 and may be re-run or polled at predetermined frequencies according to customizable system or rule settings. In one non-limiting embodiment the water quality, flow and system and rainfall conditions may be tested every 30 seconds or up to a maximum 60 seconds, with water monitoring and control thus effectively occurring in real-time.

Referring to FIG. 4, rainfall condition(s) 106, water quality and flow condition(s) 108, and/or system data condition(s) 110 are evaluated in parallel which may function to reduce informational noise while also improving responsiveness of real-time water monitoring and control system 42 and its ability to control the outcome with a great deal of precision. The real-time water monitoring and control system is thus configured to determine in parallel i) one or more of water quality and flow of the effluent based on the plurality of sensors 56 and 58 seen in FIG. 3; and ii) whether the rainfall condition has been met. System 42 may i) analyze in parallel evaluation of rainfall data and water quality and/or flow conditions; ii) analyze rainfall data first, then water quality and/or flow data; or ii) analyze water quality and/or flow data first, and rainfall data next shortly thereafter.

Therefore, real-time water monitoring and control system 42 as herein described enables monitoring of rainfall and pH and/or turbidity levels at effectively or substantially the same time and is configured to dynamically and automatically respond to changing conditions as they occur. The real-time water monitoring and control system as herein described thus enables the owner/developer of the site to selectively meet less stringent water quality requirements where/when rainfall conditions are met in real-time in a manner that complies with regulations, thereby saving the owner/developer time and costs associated with on-site water treatment. Real-time water monitoring and control system 42 as herein described may therefore provide developers with an opportunity to optimize the water treatment cost and help reduce their risk of spill-related enforcement measures, such as fines or stop work orders.

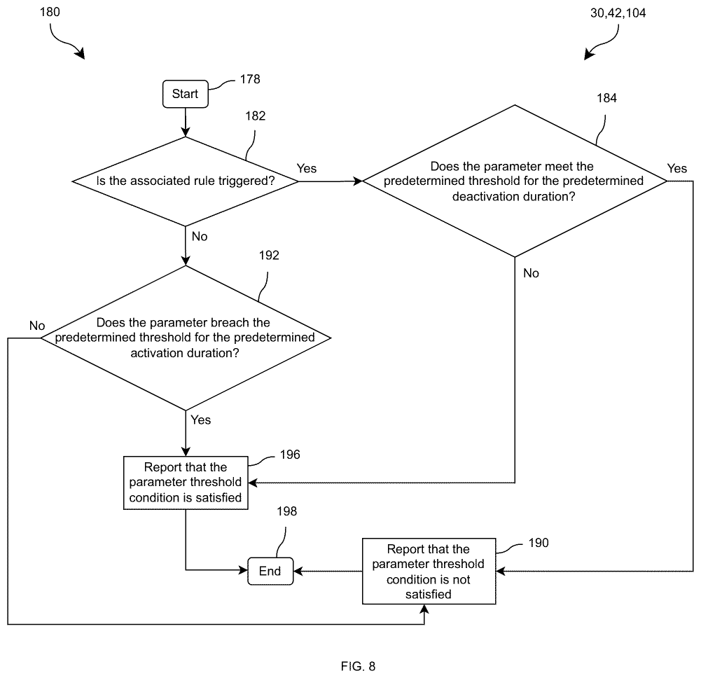

FIGS. 6 and 7 have described parameter conditions as comprising or incorporating predetermined and customizable time periods. In addition and referring to FIG. 8, real-time water monitoring and control system 42 may be said to include parameter threshold conditions for determining/customizing/adjusting activation/deactivation levels. The latter may be referred to as thresholds of activation/deactivation. A parameter threshold condition may refer to whether a parameter condition has been satisfied for one or more of predetermined activation or deactivation durations or times.

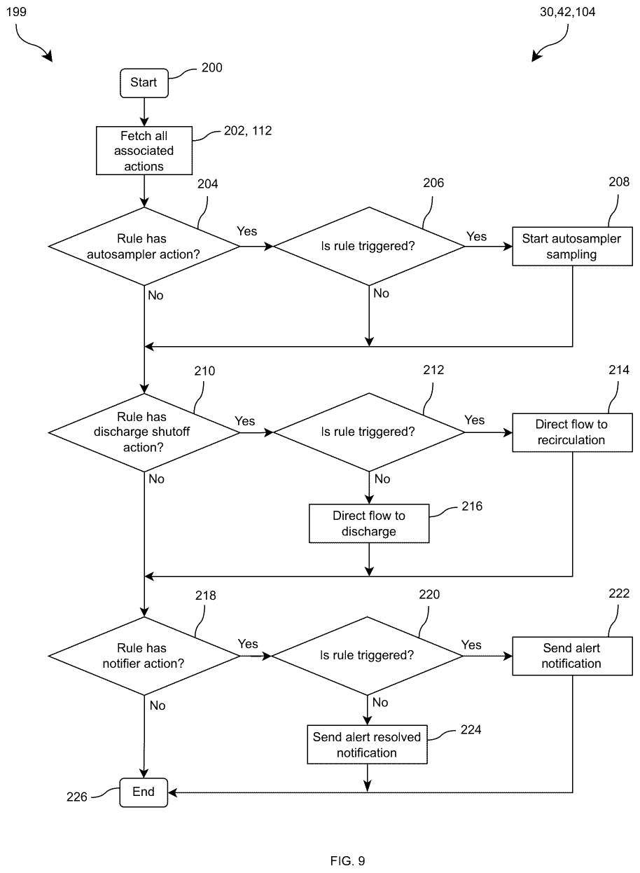

To determine whether a parameter threshold condition is satisfied, the processor starts (box 178) a parameter threshold condition evaluation process 180. As part of the process as shown by box 182, the processor or rule engine thereof determines whether an associated rule has been triggered per for example condition evaluation process 136 of FIG. 5. If so and as shown by box 184, the processor or rule engine thereof next determines whether the parameter under evaluation meets or complies with a predetermined/customizable threshold for deactivation or a deactivation duration. For example and referring to FIGS. 3 and 9, a triggered rule may comprise directing effluent to recirculate via flow device 68 upon determining that the effluent satisfied parameter condition 186 of a pH being equal to or greater than 8.7 for equal to or greater than a predetermined amount of time such as 3 minutes. The threshold for deactivation of the rule may be effluent having a pH less than 8.7 for a predetermined/customizable amount of time 188, in this non-limiting example 5 minutes. Referring back to FIG. 8 and as shown by box 190, in this case the processor or rule engine thereof reports that the parameter threshold condition is satisfied and the processor may function to stop operation of the rule and associated actions therewith. In this example, flow device 68 seen in FIG. 3 may be actuated to enable discharge of the effluent which has now been determined to have a pH of less than 8.7 for a period of time of greater than 5 minutes.

Referring back to FIG. 8, if the associated rule is not triggered and as shown by box 192, in this case the processor or rule engine thereof determines whether the parameter under evaluation breaches or does not comply with the predetermined threshold for a predetermined/customizable threshold for activation or an activation duration. For example shown, the effluent may have a pH of greater than 8.7 occurring for a time period such as 2 minutes which is less than a predetermined/customizable amount of time, in this non-limiting example 3 minutes. In this case, even though the pH level of the effluent is greater than 8.7, the rule is not triggered because the effluent has not been at this level for 3 minutes. Referring to FIG. 8 and as shown by box 190, in this case the processor or rule engine thereof reports that the parameter threshold condition is not satisfied and neither the rule nor associated actions (e.g. enabling recirculation) are triggered/activated.

If the parameter meets or complies with the predetermined threshold for at least or greater than the predetermined/customizable activation duration and as shown by box 196, in this case the processor or rule engine thereof determines that the parameter threshold condition is satisfied. In one non-limiting example, the effluent may have a pH of greater than 8.7 occurring for a period of time of at least 3 minutes, thereby causing the parameter threshold condition to be satisfied and the rule to be activated/triggered. This functions to actuate flow device 68 seen in FIG. 3 to direct flow to recirculation 49 seen in FIG. 2. Referring back to FIG. 8, the process of evaluating parameter threshold conditions thereafter reaches its end 198 and may be re-run or polled at predetermined frequencies according to customizable system or rule settings. In one non-limiting embodiment water quality may be tested every 30 seconds or up to every 60 seconds, with water monitoring and control thus effectively occurring in real-time.

Real-time water monitoring and control system 42 as herein described with its rule engine thereof therefore includes activation and deactivation rules which enable further customization thereof. Activation/deactivation thresholds and the ability to customize the same may be particularly advantageous for situations comprising storm water with rapidly and frequently changing water quality such as construction wastewater and the control thereof, for example.

In some cases and/or at some sites, water quality may be unstable and can change suddenly. Some cites may also have no water treatment system requirements. Water quality of effluent can thus change frequently with a fluctuating pH, turbidity, conductivity and/or temperature. Temperature, pH, turbidity, conductivity and the like of effluent may be subject to random or periodic fluctuations, short-term spikes and abnormalities and instantaneous/more-rapid responsiveness to the same may otherwise overload the end user, developer and/or regulatory body with notifications and/or impair proper functioning of real-time water monitoring and control system 42.

Activation/deactivation thresholds for triggering or de-triggering a rule as herein described may function to reduce system/informational noise. The activation/deactivation thresholds may additionally provide the end user, developer and/or regulatory body with a greater degree of customization to suit their needs or regulatory requirements in a manner that balances efficiency, convenience and/or cost-effectiveness. System 42 with its activation/deactivation thresholds as herein described is configured to reduce system/informational noise from such fluctuation by, for example, actuating flow device 68 seen in FIG. 3 to direct flow to recirculation 49 seen in FIG. 2 until the pH level of the effluent is within a predetermined threshold or acceptable criteria for a certain amount of predetermined/customizable time. The processor and/or rule engine thereof are thus configured to effectively reduce in part the responsiveness of real-time water monitoring and control system 42 so as to promote the occurrence of more stable upstream behavior for evaluation and discharge.

In a more variable environment, the deactivation thresholds or predetermined time within which condition(s) must be met to enable discharge of effluent, can be made to comprise a more prolonged period of time as desired/required. For example, if it is determined that pH levels at a given site are changing too frequently, real-time water monitoring and control system 42 enables the user or operator to adjust the deactivation threshold to a longer period of time: e.g. the flow device does not enable discharge unless pH levels are within water specification for more than 5 minutes (instead of a previous threshold of 3 minutes).

Combining different conditions and different activation/deactivation durations may function to enable the end user, operator and/or regulatory body to achieve a clean and predictive outcome while also reducing system/informational noise. The activation/deactivation thresholds may also enable real-time water monitoring and control system 42 to comply with sophisticated legislation, for example.

The customization of real-time water monitoring and control system 42 enables the rule engine thereof to be altered in real-time by the user and/or regulatory body and this provides the system with yet further flexibility, such as for catastrophic or severe weather events. This may be particularly advantageous as with climate change, catastrophic weather events, such as flooding or atmospheric rivers, may be occurring more often. In this case, real-time water monitoring and control system 42 is configured (by the end user, operator and/or regulatory body) to enable discharge at further tiered levels of water quality to reflect the same and/or also take into the potential environmental/social damage occurring from such catastrophic events and wanting to mitigate against the same. Thus, instead of two tiers of water quality criteria based on whether a significant rainfall event has occurred, the system with its rule engine thereof enables the creation of additional tiers of water quality criteria based on multiple rainfall thresholds. As a non-limiting example: a level 1 significant rainfall event may comprise 25 mm of rainfall over 24 hours with effluent having a turbidity level of 100 NTU being acceptable for discharge in this case; a level 2 significant rainfall event may comprise 50 mm of rainfall over 24 hours with effluent having a turbidity level of 125 NTU being acceptable in this case; and a level 3 significant rainfall event may comprise 100 mm of rainfall over 24 hours with effluent having a turbidity level of 200 NTU being acceptable for discharge in this case. System 42 as herein described is thus configured to adjust acceptable water quality thresholds for the effluent in real-time depending on rainfall data so determined.

On the other hand and referring to FIG. 1, when site 31 is continually recirculating its water over a prolonged period of time, water levels on the site can rise and this can be an undesirable situation for a developer or site owner. Thus when a valve is closed for a period of time, recirculating of water at the site may be a big deal.

System 42 may include or be customized to include notification actions associated with one or more rules and which notify a user if recirculation is occurring and/or a flow device is closed at various predetermined and user customizable time periods, such as at 5 min, 15 min, 60 min etc. The notification system may be useful as if system 42 otherwise sends notification every time a valve is closed, this may be annoying and result in a lot of system/informational noise. The notification system in this example is configured to include a hierarchy of escalating severity levels of notifications: e.g. info notifier, warning notifier, danger notifier, critical notifier etc. and these may be associated with where discharging of effluent has been inhibited/shut-off, in one non-limiting example. System 42 is also customizable to allow each user receive various types and severity levels of notifications depending on the rule(s) and/or user settings such as: a site superintendent who may get various levels of notifications e.g. warning, danger and critical alerts about recirculation and loss of power to the system 42 (e.g. set at 15 min, 60 min, 3 hours); a project manager who may get only critical alerts about recirculation e.g. at 3 hours; a regulatory official who receive all alerts and/or only critical alerts etc. System 42 as herein described may thus provide a notification system that is layered and customizable to each user according to their role on site 31 which may function to reduce system/informational noise and inhibit the prospects of emergency and inadvertent discharge of effluent which is outside of given water specifications.

The customization of real-time water monitoring and control system 42 thus enables the developer/owner to seek to comply with regulatory requirements on the one hand in real-time in a manner that manages informational noise while tailoring activation/deactivation thresholds to maximize discharge and minimize recirculation.

System 42 as herein described is flexible in regards to both parameter evaluation and rainfall conditions. For example, the system not only determines if rainfall data exceeds a predetermined threshold for a period of time, but also determines if that event had occurred in a specific period of time following the initial detection. System 42 further includes and/or provides the ability to set or customize different activation/deactivation durations and the like, for example.

FIG. 9 shows a non-limiting embodiment of a rule actions process 199 or processing flowchart for real-time water monitoring and control system 42 as herein described. The processor starts (box 200) the process by first fetching all associated actions 202. The process in this non-limiting embodiment first determines where the rule has an autosampler or automated sampling device action as shown by box 204. If yes, the process next determines whether the associated rule is triggered as shown by box 206 and if so the processor is configured to initiate autosampler sampling as shown by box 208.

In either case the processor next determines whether the rule has a discharge shutoff or flow device action as shown by box 210. If yes, the processor determines whether the associated rule is triggered as shown by box 212 and if so the processor directs effluent flow to recirculation as shown by box 214, else the processor directs the effluent flow to discharge as shown by box 216.

In either case the processor next determines whether the rule has a notifier or notification action as shown by box 218. If yes, the processor determines whether the associated rule is triggered as shown by box 220 and if so sends an alert notification as shown by box 222, else the processor sends an alert resolved notification as shown by box 224. Rule actions process 199 thereafter reaches its end 226 and may be re-run or polled at predetermined frequencies according to customizable system or rule settings. The above processing order is not strictly required and may be conducted in other orders and/or in parallel in other embodiments, for example.

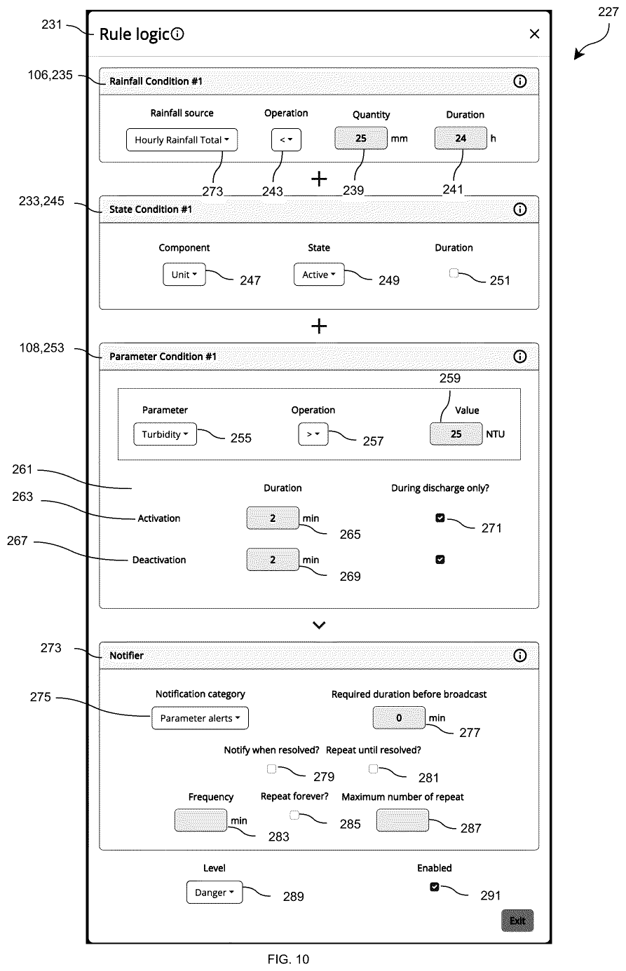

FIG. 10 is a non-limiting example of a user interface 227 for creating, adjusting and/or customizing a rule 231 for the rule engine of real-time water monitoring and control system 42. The user interface may be a menu-driven user interface in this example, though this is not strictly required. User interface 227 may be referred to as a rule creation and modification interface. The user interface may enable any number of conditions to be added in the rule creation, such as one or more rainfall conditions 106, a system state condition 233, and one or more parameter conditions such as water quality and flow condition 108. For the rainfall condition, a rainfall section 235 of user interface 227 is provided in which the end user can specify rainfall source 237 of the rainfall data may be selected, such as an hourly rainfall total as obtained by third party weather API 92 seen in FIG. 4. Referring back to FIG. 10, the rainfall section of the user interface further enables quantity 239 and duration 241 of the rainfall to be entered according to customized and/or tailored requirements for the jurisdiction/project in question: in this non-limiting example 25 mm for 24 hours is specified. Rainfall section 235 of user interface 227 enables the end-user, developer and/or regulatory body to specify a comparison operator 243 (i.e. less than, greater than, or equal to), with in this case the rainfall condition being set to the rainfall total needing to be equal to or less than 25 mm within the last 24 hours in order to meet rainfall condition 106.

For state condition 233, a state section 245 of user interface 227 is provided in which the end user can specify the component 247 (e.g. a valve, pump etc.) and a state 249 thereof (e.g. active, under maintenance, inactive etc.), as well as a duration 251 for the same. One non-limiting example of a state condition may be: is unit 52 determined to be in an active state? If so, this condition is satisfied.

For water quality and flow condition 108, a parameter section 253 of user interface 227 is provided in which the end user can specify the parameter 255 to be tested (e.g. turbidity, pH level, temperature etc.), a comparison operator 257 (i.e. less than, greater than, or equal to) and the value of the parameter (e.g. 25 NTU). In one non-limiting example, water quality and flow condition 108 may thus comprise: is the turbidity of the effluent greater than or equal to 25 NTU? If so the condition is satisfied.

User interface 227 may also enable parameter threshold or activation/deactivation conditions 261 to be added in or part of the rule creation. An activation section 263 of the user interface is provided in which the end user can specify a first predetermined period of time or duration 265 (e.g. 2 minutes) within which one or more of conditions 106, 108 and 233 must be true, in this example, water quality and flow condition 108, in order the activation condition to be satisfied for triggering the rule e.g. if there is less than 25 mm of rain within the past 24 hours, the unit state is active (i.e. not in a maintenance or other non-operational state) and the turbidity level of the effluent is equal to or greater than 25 NTU for over 2 minutes, then the rule is triggered and flow device 68 seen in FIG. 3 is actuated by real-time water monitoring and control system 42 to recirculate the effluent rather than to discharge the same. The unit state feature may ensure that where the unit is in maintenance, alerts are not issued during this time, for example.

A deactivation section 267 of the user interface is provided in which the end user, developer and/or regulatory body can specify a second predetermined period of time or duration 269 (e.g. 2 minutes) within which the associated condition (in this case water quality and flow condition 108) must no longer be true in order for the deactivation condition to be satisfied. Thus, regardless of rainfall condition 106 and state condition 233 which have no activation/deactivation conditions associated therewith in this non-limiting example of a rule, if real-time water monitoring and control system 42 determines that the turbidity level of the effluent is less than 25 NTU for over 2 minutes, then the deactivation threshold has been met, the rule is no longer triggered and flow device 68 seen in FIG. 3 is actuated by real-time water monitoring and control system 42 to enable discharge of effluent once more. There may be an additional rule or parameter condition in this case which incorporates or considers pH levels of the effluent, for example.

Activation and deactivation sections 263 and 267 of user interface 227 further enable the end-user, developer and/or regulatory body to specify that the activation/deactivation conditions are met during discharge only 271: i.e. when a discharge flow is detected or determined to occur. Thus, if the rule when triggered is supposed to direct effluent to recirculate but real-time water monitoring and control system 42 determines that there is no flow of effluent through water treatment system 34 seen in FIG. 1 and/or no flow through the water monitoring and control system, in this case this feature causes the water flow and control system to not bother trying to recirculate non-flowing effluent.

User interface 227 includes in this example a notifier section 273 that enables notification action(s) 116 seen in FIG. 4 and associated with triggered rules to be customized. Referring back to FIG. 10, the notifier section of the user interface may enable a notification category 275 (e.g. parameter alert) to be specified, together with a predetermined time period or direction 277 before the notification is broadcast. Notifier section 273 of user interface 227 includes in this example an ability to specify whether a notification should be sent upon the alert/associated-rule being resolved, as shown by arrow 279, as well as the ability to specify whether the alert should be repeated until the alert/associated-rule has been resolved as shown by arrow 281. If the end-user, operator and/or regulatory body would like the alert repeated, user interface 227 enables specification of the frequency of repetition 283 (i.e. an alert per X minutes), the ability for the alert to repeat forever 285 as well the ability to specify a maximum number of repeats of the alert as shown by arrow 287. Notifier section 273 of user interface 227 in this non-limiting embodiment includes the ability to specify the level type 289 of the alert (e.g. warning, danger, critical etc.) if enabled 291. In addition or alternatively, the latter may enable the end user, operator and/or regulatory body to disable the alert feature.

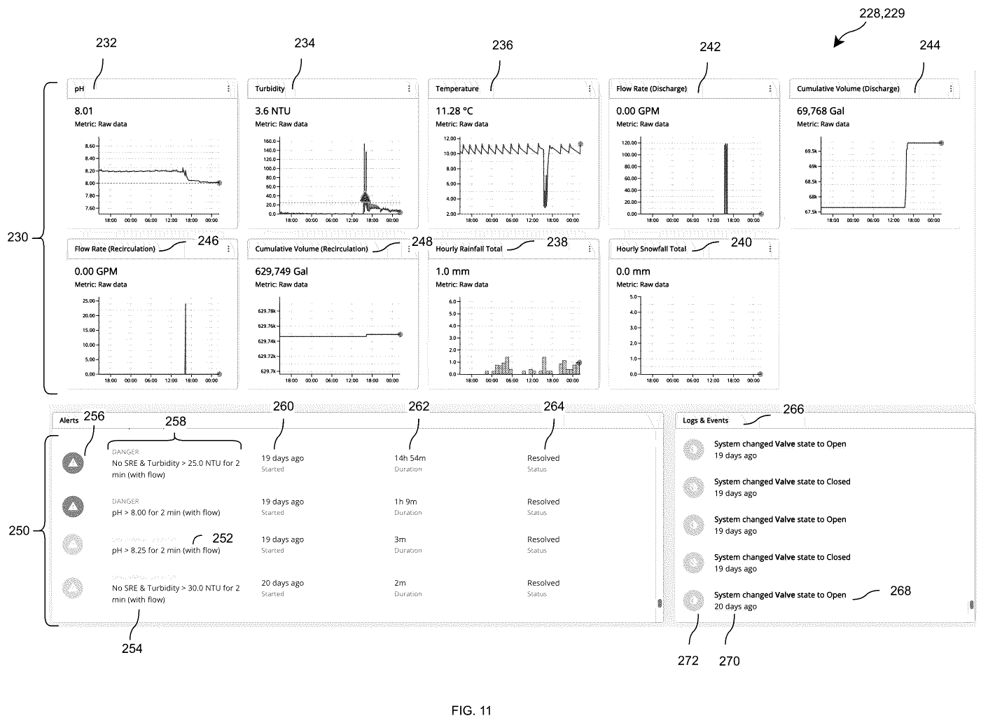

As seen in FIG. 11, real-time water monitoring and control system 42 may further be configured to include a dashboard interface 228. The system may also include a computer display device 229 via which the dashboard interface may be displayed, which may be part of and/or coupled to a laptop, tablet and/or smart phone for example. Dashboard interface 228 may be a part of a graphical user interface, but though this is not strictly required.

The dashboard interface in this example includes a data visualization section 230 which displays in real-time raw water quality and rainfall data so obtained from real-time water monitoring and control system 42. This section of dashboard interface 228 may display information in individual graph form or via a plurality of graphs shown on one display for example as shown in FIG. 11. In this non-limiting example, data visualization section 230 of dashboard interface 228 includes: a graph 232 of PH levels of effluent as a function time, a graph 234 of turbidity levels of the effluent as a function of time, a graph 236 of temperature levels of the effluent as a function of time, a graph 238 of rainfall amount totals as a function of time and a graph 240 of snowfall amount totals as a function of time.

Data visualization section 230 of dashboard interface 228 in this example includes flow data of real-time water monitoring and control system 42 displayed in real-time. In this non-limiting example, the data visualization section of the dashboard interface includes: a graph 242 of the discharge flow rate of effluent through the system as a function of time, a graph 244 of the corresponding cumulative discharge volume through the system, a graph 246 of the flow rate of effluent that is recirculated instead of discharged based on decisions made by the system's rule engine, a graph 248 of the corresponding cumulative volume of the effluent that is recirculated instead of discharged. The time axis interval for each of graphs 232, 234, 236, 238, 240, 242, 244, 246 and 248 may be customizable and in this non-limiting embodiment the graphs display data continuously via and spanning an hourly scale. The dashboard of real-time water monitoring and control system 42 may enable the owner, end-user and/or regulatory body to readily and precisely identify when flow device 68 seen in FIG. 3, such as a valve or pump, was triggered for example. Data visualization section 230 of dashboard interface 228 may enable an end-user, operator and/or regulatory body to identify system behavior patterns and adjust/customize the rule engine of real-time water monitoring and control system 42 in response thereto: for example, depending on site dynamics one may want to change the rules to avoid issuing alerts and/or activating recirculation every 10 minutes etc. Monitoring the deactivation pattern may also be helpful/important at reducing system/informational noise.

Referring back to FIG. 11, dashboard interface 228 in this example includes an alert information section 250. The alert information section in this non-limiting embodiment may including different types of alerts such as danger parameter alerts, system state alerts and discharge shutoff alerts. A non-limiting example of a warning alert 252 is where a rule is triggered in which real-time water monitoring and control system 42 determines that there is no significant rain event and determines that the turbidity level of the effluent is greater than 25 NTU for more than 2 minutes with flow of effluent being detected, in which case the system continues to discharge effluent and signals/sends/sounds this warning alert. A non-limiting example of a discharge shutoff alert 254 is where a rule is triggered in which real-time water monitoring and control system 42 determines that there is no significant rain event and determines that the turbidity level of the effluent is greater than 30 NTU for more than 2 minutes with flow of effluent being detected, in which case the system stops discharging effluent and signals/sends/sounds this discharge shutoff alert.

Another non-limiting example of a warning alert is where a rule is triggered in which the system determines that the system determines that the pH level of the effluent is greater than 8.5 for more than 2 minutes, in which case the system continues to discharge effluent and signals/sends/sounds the warning alert. Another non-limiting example of a discharge shutoff alert is where a rule is triggered in which the system determines that the system determines that the pH level of the effluent is greater than 8.5 for more than 10 minutes, in which case the system stops discharging of effluent and signals/sends/sounds this discharge shutoff alert.

Alert information section 250 of dashboard interface 228 in this non-limiting example includes an alert type indication or icon 256 (i.e. warning alert, discharge shutoff alert etc.), a rule description 258 associated with the alert (such as those set out above), a time stamp or indication 260 as to when the alert first occurred, an indication of the duration 262 of the alert and an indication of whether the rule associated with the alert was resolved 264.

Dashboard interface 228 in this non-limiting example includes a log and events section 266, which may alternatively be referred to as the log section. This section is configured to display logs of all system events, status updates and the like in chronological order. Log and events section 266 may include log entries 268 such as “system changed its unit state to active” when real-time water monitoring and control system 42 is turned on, “system changed its unit state to offline” when the system is turned off, “system changed valve state to open” when the system determines that effluent may be discharged etc. Such log entries include a time stamp or indication 270 as to when they occurred. Log and events section 266 may additionally include a log type indication or icon 272 (e.g. a Wi-Fi® icon to symbolize turning on/off of the system, a water drop icon to symbolize a valve opening, a wrench icon to symbolize a manual override or repair of the system etc.) though this is not strictly required. The log and events section may thus function to provide the end-user, operator and/or regulatory body with a quick snapshot of the latest status of real-time water monitoring and control system 42 or events thereof.

Many additional advantages result from the structure of the present invention. System 42 as herein described is configured to function with any municipality. The system as herein described is designed to be inherently flexible with the ability to adapt to different municipal regulations. System 42 as herein described is also customizable to adapt to or work in conjunction with different water treatment systems. For example, some treatment systems are configured to discharge effluent in batches, e.g. every 30 minutes whereas others may be configured to discharge for 2 or 5 minutes, then stop for an undefined period of time and repeat with frequency dictated by the volume and rate of the influent. The customization of the rule engine enables real-time water monitoring and control system 42 to readily adapt to or be used in conjunction with such a variety of water treatment system configurations via various templates that may be tailored/tweaked as required to achieve a reliable and predictable behaviour, while minimizing the information noise and volume of effluent discharged outside of specifications. The system is configured such that whenever a change is made to one of the rules of the rule engine thereof, a change log or record is created and saved in memory of the system to provide full transparency and auditability of the data thereof.

The activation/deactivation rules which are customized based on predetermined time thresholds, may function to provide a system that is further nuanced while inhibiting excess signal noise.

System 42 as herein described operates dynamically and in real-time including incorporating rainfall data in parallel with water quality and flow data. This may be advantageous because onsite construction water treatment is a dynamic process and its effectiveness may vary: water quality of the effluent may decline drastically in a relatively short period of time (e.g. a pH may spike from 7.5 to 11.1 in an hour, or a turbidity may increase from 22 NTU to 120 NTU in just 2 minutes) due to multiple factors such as i) operational failure of the water treatment system, ii) a sudden change in the water source composition and/or iii) insufficient/flawed design of the water treatment system.

Remote server 86 of real-time water monitoring and control system 42 seen in FIG. 3 may function as a safeguard against compromised or manipulated results. The cloud-like/remote aspect of the server (and/or components/data thereof) including the record of system and rule changes, log of issued and resolved alerts and state change events (e.g. power loss, maintenance) and other features described herein may function to facilitate monitoring of onsite water treatment system 34 seen in FIG. 2 by a neutral, arms-length provider in an independent and transparent manner.

System 42 as herein described may comprise application-specific and compliance-driven software that tracks and analyzes a wide range of water quality, flow and operational parameters in effectively real-time while also enabling effective control/direction of effluent that is responsive, dynamic and effectively in real-time via communication between remote server 86 and onsite unit 52. The software is configured to work in sync with the hardware of the onsite unit to enable flow re-routing of effluent based on any combination of conditions, including dynamic parameters such as rainfall, to readily comply even with highly-stringent/complex regulations. System 42 as herein described may thus comprise an improved fail-safe means of preventing the release of non-compliant water into the environment.

System 42 as herein described may be said to comprise a computer program product including a medium carrying computer readable instructions which, when executed by processor 82 seen in FIG. 3, cause the processor to execute one or more of the processes herein described. Server 86 as herein described may comprise said computer program product, with the server being configured to deliver the computer readable instructions to a recipient computer by a way of a communication medium. The terms “medium” and “program product” are intended to be non-limiting in scope and may comprise any transmission-type media.

It will be appreciated that many variations are possible within the scope of the invention described herein. For example, real-time water monitoring and control system 42 as herein described has been discussed primarily in relation to site 31 seen in FIG. 1 such as a construction or development site. However, this is not strictly required and the system may be used in other environments, such as for example real-time monitoring of sewer systems or public bodies of water. The latter may include monitoring the health of urban streams, with the data obtained therefrom and displayed on dashboard interface 228 seen in FIG. 11 facilitating real-time research and feedback that may function to i) inform future policy decisions, ii) assist in determining the effectiveness of regulations and/or iii) identify pollutants/sources-of-pollution.

Where a component (e.g. a software module, processor, assembly, device, circuit, etc.) is referred to herein, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Embodiments of the invention may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a control circuit for a device may implement methods as described herein by executing software instructions in a program memory accessible to the processors.

Processing may be centralized or distributed. Where processing is distributed, information including software and/or data may be kept centrally or distributed. Such information may be exchanged between different functional units by way of a communications network, such as a Local Area Network (LAN), Wide Area Network (WAN), or the Internet, wired or wireless data links, electromagnetic signals, or other data communication channel.

The invention may also be provided in the form of a program product. The program product may comprise any non-transitory medium which carries a set of computer-readable instructions which, when executed by a data processor, cause the data processor to execute a method of the invention. Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.

In some embodiments, the invention may be implemented in software. For greater clarity, “software” includes any instructions executed on a processor, and may include (but is not limited to) firmware, resident software, microcode, code for configuring a configurable logic circuit, applications, apps, and the like. Both processing hardware and software may be centralized or distributed (or a combination thereof), in whole or in part, as known to those skilled in the art. For example, software and other modules may be accessible via local memory, via a network, via a browser or other application in a distributed computing context, or via other means suitable for the purposes described above.

Software and other modules may reside on servers, workstations, personal computers, tablet computers, and other devices suitable for the purposes described herein.

### Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms. These terms (“a”, “an”, and “the”) mean one or more unless stated otherwise;
“and/or” is used to indicate one or both stated cases may occur, for example A and/or B includes both (A and B) and (A or B);
“approximately” when applied to a numerical value means the numerical value±10%;
where a feature is described as being “optional” or “optionally” present or described as being present “in some embodiments” it is intended that the present disclosure encompasses embodiments where that feature is present and other embodiments where that feature is not necessarily present and other embodiments where that feature is excluded. Further, where any combination of features is described in this application this statement is intended to serve as antecedent basis for the use of exclusive terminology such as “solely,” “only” and the like in relation to the combination of features as well as the use of “negative” limitation(s)” to exclude the presence of other features; and
“first” and “second” are used for descriptive purposes and cannot be understood as indicating or implying relative importance or indicating the number of indicated technical features.

Words that indicate directions such as “vertical”, “transverse”, “horizontal”, “upward”, “downward”, “forward”, “backward”, “inward”, “outward”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Where a range for a value is stated, the stated range includes all sub-ranges of the range. It is intended that the statement of a range supports the value being at an endpoint of the range as well as at any intervening value to the tenth of the unit of the lower limit of the range, as well as any subrange or sets of sub ranges of the range unless the context clearly dictates otherwise or any portion(s) of the stated range is specifically excluded. Where the stated range includes one or both endpoints of the range, ranges excluding either or both of those included endpoints are also included in the invention.

Certain numerical values described herein are preceded by “about”. In this context, “about” provides literal support for the exact numerical value that it precedes, the exact numerical value±5%, as well as all other numerical values that are near to or approximately equal to that numerical value. Unless otherwise indicated a particular numerical value is included in “about” a specifically recited numerical value where the particular numerical value provides the substantial equivalent of the specifically recited numerical value in the context in which the specifically recited numerical value is presented. For example, a statement that something has the numerical value of “about 10” is to be interpreted as: the set of statements:
in some embodiments the numerical value is 10;
in some embodiments the numerical value is in the range of 9.5 to 10.5;
   and if from the context the person of ordinary skill in the art would understand that values within a certain range are substantially equivalent to 10 because the values with the range would be understood to provide substantially the same result as the value 10 then “about 10” also includes:
in some embodiments the numerical value is in the range of C to D where C and D are respectively lower and upper endpoints of the range that encompasses all of those values that provide a substantial equivalent to the value 10

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

As will be apparent to those of skill in the art upon reading this disclosure, each of the individual embodiments described and illustrated herein has discrete components and features which may be readily separated from or combined with the features of any other described embodiment(s) without departing from the scope of the present invention

Any aspects described above in reference to apparatus may also apply to methods and vice versa.

Any recited method can be carried out in the order of events recited or in any other order which is logically possible. For example, while processes or blocks are presented in a given order, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, simultaneously or at different times.

Various features are described herein as being present in “some embodiments”. Such features are not mandatory and may not be present in all embodiments. Embodiments of the invention may include zero, any one or any combination of two or more of such features. All possible combinations of such features are contemplated by this disclosure even where such features are shown in different drawings and/or described in different sections or paragraphs. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible). This is the case even if features A and B are illustrated in different drawings and/or mentioned in different paragraphs, sections or sentences.

### Additional Description

System 42 as herein described may function to make it easier for construction sites and the like discharge effluent or runoff into the storm sewer system/environment to achieve compliance throughout the seasons. Some jurisdictions allow for different regulatory criteria for acceptable water quality of the site effluent: stricter criteria applied under so called normal weather conditions and more relaxed criteria applied during wet (or extreme rainfall) conditions. Wet conditions could be defined following a set of specified criteria (e.g. total rainfall received in one hour, 24 hours, etc. is greater than a specified amount) and could change with seasons and/or jurisdictions. It may however be challenging to manually verify in real time whether the wet conditions are satisfied to allow for compliance with more lenient criteria. It may equally be challenging to manually verify in real time whether the wet conditions are no longer satisfied and immediately switch to compliance with stricter criteria, making it very challenging to stay compliant while trying to take advantage of this benefit that has a significant commercial value.

System 42 as herein described enables site owners, developers and the like to discharge under more relaxed compliance criteria and this may owners/developers of a construction site or the like to decrease their water treatment cost by utilizing a smaller system as well as save footprint on site which is often valued at a premium. However, non-compliance (e.g. discharging under relaxed criteria when the wet conditions are no longer satisfied) can lead to fines, penalties and stop work orders resulting in project delays and increased costs. System 42 as herein described may thus provide the ability to ensure strict and continuous compliance of construction site effluent with applicable regulations while taking full advantage of this benefit would likely result in cost savings for the construction site.

Examples of real-time water monitoring control systems and processes have been described. The following clauses are offered as further description.
(1) A real-time water monitoring and control process comprising: obtaining rainfall data in real-time via one or more of a rain gauge and a weather application programming interface (API) and measuring water quality, flow and system data in real-time via one or more sensors; determining in real time via a processor whether the rainfall data satisfies a set of one or more predetermined rainfall conditions; in parallel or sequentially, in any order, determining in real time whether one or more of the water quality, flow and system data satisfies a set of one or more of predetermined conditions, and if so, sending a signal to activate one or more of a notification, a water flow device and an automated sampling device; and sending a signal to deactivate all triggered actions when any said predetermined condition is no longer satisfied.
(2) A real-time water monitoring and control process according to clause 1 or any other clause herein, wherein for the one or more predetermined rainfall conditions, determining whether the rainfall amount calculated based on a rolling window of a predetermined duration with an end point being the current moment exceeds a predetermined threshold or whether the rainfall amount exceeded the predetermined threshold at an earlier point in time but within another predetermined duration with the end point being the current moment.
(3) A real-time water monitoring and control process according to clause 1 or any other clause herein, wherein for the one or more predetermined rainfall conditions, determining whether the rainfall amount calculated based on a rolling window of a predetermined duration with an end point being the current moment exceeds a predetermined threshold and whether the rainfall amount exceeded the predetermined threshold at an earlier point in time but within another predetermined duration with the end point being the current moment.
(4) A real-time water monitoring and control process according to any preceding clause or any other clause herein, wherein for the one or more predetermined water quality, flow and system data conditions, determining whether one or more of the water quality, flow and system data satisfy a predetermined comparison operator (less than, greater than or equal to) when evaluated against a predetermined threshold thereof for a predetermined duration thereof with the end point being the current moment.
(5) A real-time water monitoring and control process of according to any preceding or subsequent clause, wherein the weather API is configured to automatically and continually query third party rainfall data based on location and time parameters.
(6) A real-time water monitoring and control process of according to any preceding clause, wherein the water flow device comprises a valve.
(7) A real-time water monitoring and control process of according to any preceding clause, wherein the water flow device comprises a pump.
(8) A real-time water monitoring and control system comprising: one or more sensors via which water quality, flow and system data is measured; one or more of rain gauge and a weather application programming interface (API) to obtain rainfall data in real-time; and a processor configured to determine in real time whether the rainfall data satisfies a set of one or more predetermined rainfall conditions and in parallel or sequentially, in any order, configured to determine in real time whether one or more of the water quality, flow and system data satisfies a set of one or more of predetermined conditions, and if so, sending a signal to activate one or more of a notification, a water flow device and an automated sampling device and sending a signal to deactivate all triggered actions when any of said predetermined conditions is no longer satisfied.
(9) A real-time water monitoring and control system according to clause 8 or any other clause herein, wherein for the one or more predetermined rainfall conditions, the processor is configured to: i) determine whether the rainfall amount calculated based on a rolling window of a predetermined duration with an end point being the current moment exceeds a predetermined threshold; or ii) whether the rainfall amount exceeded the predetermined threshold at an earlier point in time but within another predetermined duration with the end point being the current moment.
(10) A real-time water monitoring and control system according to clause 8 or any other clause herein, wherein for the one or more predetermined rainfall conditions, the processor is configured to: i) determine whether the rainfall amount calculated based on a rolling window of a predetermined duration with an end point being the current moment exceeds a predetermined threshold; and ii) whether the rainfall amount exceeded the predetermined threshold at an earlier point in time but within another predetermined duration with the end point being the current moment.
(11) A real-time water monitoring and control system according to any one of clauses 8 to 10 or any other clause herein, wherein for the one or more predetermined water quality, flow and system data conditions, the processor is configured to determine whether one or more of the water quality, flow and system data satisfy a predetermined comparison operator (less than, greater than or equal to) when evaluated against a predetermined threshold thereof for a predetermined duration thereof with the end point being the current moment.
(12) A real-time water monitoring and control system of according to any one of clauses 8 to 11 or any clause herein, wherein the water flow device comprises a valve.
(13) A real-time water monitoring and control system of according to any one of clauses 8 to 11 or any clause herein, wherein the water flow device comprises a pump.
(14) A real-time water monitoring and control process of according to any one of clauses 8 to 13 or subsequent clause, wherein the weather API is configured to automatically and continually query third party rainfall data based on location and time parameters.
(15) A real-time water monitoring and control process comprising: obtaining rainfall data in real-time and measuring water quality, flow and system data in real-time via one or more sensors; determining via a processor whether the rainfall data exceeds one or more predetermined thresholds; if the rainfall data exceeds one or more predetermined thresholds, the process includes determining whether one or more of the water quality, flow and system data exceeds a first set of one or more predetermined thresholds and if so, sending a first signal to activate one or more of a first notification and a water flow device; if the rainfall data is below the one or more predetermined thresholds, the process includes determining whether one or more of the water quality, flow and system data exceeds a second set of one or more predetermined thresholds and if so, sending a second signal to activate one or more of a second notification and said water flow device.
(16) A real-time water monitoring and control process according to clause 15 or any other clause herein, wherein the process includes determining whether the rainfall data exceeds said one or more predetermined thresholds for a first predetermined period of time and if so, the process includes determining whether said one or more of the water quality, flow and system data exceeds the first set of one or more predetermined thresholds for one or more predetermined periods of time and if so, the process includes sending the first signal to activate one or more of said first notification and the water flow device.
(17) A real-time water monitoring and control process according to clause 16 or any other clause herein, wherein if the rainfall data is below the one or more predetermined thresholds for the first predetermined period of time, the process includes determining whether one or more of the water quality, flow and system data exceeds a second set of one or more predetermined thresholds for one or more predetermined periods of time and if so, the process includes sending the second signal to activate one or more of said second notification and said water flow device.
(18) A real-time water monitoring and control process according to any one of clauses 15 to 17 or any clause herein, wherein one or more of the first signal activates an automated sampling device and the second signal activates the automated sampling device.
(19) A real-time water monitoring and control system comprising: one or more sensors configured to measure water quality, flow and system data in real-time; a rainfall acquisition device configured to obtain rainfall data in real-time; and a processor configured to determine whether the rainfall data exceeds one or more predetermined thresholds, whereby if the rainfall data exceeds one or more predetermined thresholds, the processor is configured to determine whether one or more of the water quality, flow and system data exceeds a first set of one or more predetermined thresholds and if so, the processor is configured to send a first signal to activate one or more of a first notification and a water flow device and whereby if the rainfall data is below the one or more predetermined thresholds, the processor is configured to determine whether one or more of the water quality, flow and system data exceeds a second set of one or more predetermined thresholds and if so, the processor is configured to send a second signal to activate one or more of a second notification and said water flow device.
(20) A real-time water monitoring and control system according to clause 19 or any other clause herein, wherein the processor is configured to determine whether the rainfall data exceeds said one or more predetermined thresholds for a first predetermined period of time and if so, the processor is configured to determine whether said one or more of the water quality, flow and system data exceeds the first set of one or more predetermined thresholds for one or more predetermined periods of time and if so, the processor is configured to send the first signal to activate one or more of said first notification and the water flow device.
(21) A real-time water monitoring and control system according to clause 20 or any other clause herein, wherein if the rainfall data is below the one or more predetermined thresholds for the first predetermined period of time, the processor is configured to determine whether one or more of the water quality, flow and system data exceeds a second set of one or more predetermined thresholds for one or more predetermined periods of time and if so, the processor is configured to send the second signal to activate one or more of said second notification and said water flow device.
(22) A real-time water monitoring and control system according to any one of clauses 19 to 21 or any clause herein, including an automated sampling device, with one or more of the first signal activating the automated sampling device and the second signal activating the automated sampling device.
(23) A real-time water monitoring and control system comprising: a plurality of sensors configured to measure properties of the water in real-time; and a processor in communication with the sensors and rainfall data in real-time and causing the water to discharge based on whether the water is below a first water quality threshold or a second water quality threshold where a rainfall condition has been met.
(24) A real-time water monitoring and control system of clause 23 or any clause herein, wherein the processor is configured to determine, in parallel or sequentially in any order, i) one or more of water quality and flow of the water based on the plurality of sensors; and ii) whether the rainfall condition has been met.
(25) A real-time water monitoring and control system of clause 23 or any clause herein, wherein the second water quality threshold is higher than the first water quality threshold.
(26) A real-time water monitoring and control system of clause 23 or any clause herein, wherein the processor determines the water quality of the water via water quality said sensors and wherein the processor determines the flow of the water via one or more flow said sensors.
(27) A real-time water monitoring and control system of clause 23 or any clause herein, wherein the processor inhibits discharge of the water where the rainfall condition has not been met and the water quality of the water as determined by the sensors exceeds the first water quality threshold and wherein the processor inhibits discharge of the water where the rainfall condition has been met and the water quality of the water as determined by the sensors exceeds the second water quality threshold.
(28) A real-time water monitoring and control system of clause 23 or any clause herein, wherein the processor recirculates the water through a water treatment system where the rainfall condition has not been met and the water quality of the water as determined by the sensors exceeds the first water quality threshold and wherein the processor recirculates the water through a water treatment system where the rainfall condition has been met and the water quality of the water as determined by the sensors exceeds the second water quality threshold.
(29) A real-time water monitoring and control system of clause 23 or any clause herein, including one or more of: a rainfall gauge via which the rainfall data is obtained; and a weather application programming interface (API) to obtain rainfall data in real-time based on a given location and time.
(30) A method of automatically monitoring and controlling water, the method comprising: obtaining rainfall data in real-time; determining via a processor whether the rainfall data for a first predetermined amount of time exceeds a predetermined threshold and if so recording that a new significant rainfall event has occurred and determining that a rainfall condition has been met, if the processor determines that the rainfall data for the first predetermined amount of time does not exceed the predetermined threshold, determining via the processor whether a previous significant rainfall event was recorded and if no, determining that the rainfall condition has not been meet; if the processor determines that the rainfall data for the first predetermined amount of time does not exceed the predetermined threshold and determines that the previous significant rainfall event was recorded, determining via the processor whether the previous significant rainfall event was recorded at a time equal to or less than a second predetermined amount of time and if so, determining that the rainfall condition has been met; if the processor determines that the rainfall data for the first predetermined amount of time does not exceed the predetermined threshold, determines that the previous significant rainfall event was recorded, and determines that the previous significant rainfall event was recorded at a time greater than the second predetermined amount of time, then determining via the processor that the rainfall condition has not been met; and determining whether water is to be discharged based on different water quality criteria depending on whether the rainfall condition has been met.
(31) A method of automatically monitoring and controlling water according to clause 30 or any clause herein, wherein the rainfall data is obtained via one or more of a rainfall gauge and a weather application programming interface (API) configured to obtain rainfall data in real-time based on a given location and time.
(32) A method of automatically monitoring and controlling water according to any one of clauses 30 to 31 or any clause herein, wherein the water quality is determined via one or more water quality and flow sensors.
(33) A water monitoring and control process comprising: continuously monitoring water at a construction site having wastewater; automatically sending a first signal to activate one or more control processes upon determining that one or more of water quality, flow and system data exceeds one or more predetermined value thresholds for greater than a first predetermined amount of time; and automatically sending a second signal to deactivate the one or more control processes upon determining the one or more of water quality, flow and system data is below the one or more predetermined value thresholds for greater than a second predetermined amount of time.
(34) A real-time water monitoring and control process according to clause 33 or any clause herein, including determining the one or more of water quality, flow and system data via one or more water quality and flow sensors.
(35) A real-time water monitoring and control process according to any one of clauses 33 to 34 or any clause herein, including continuously monitoring rainfall data at or adjacent the construction site and using said rainfall data in real-time to determine the one or more predetermined value thresholds against which the water quality, flow and system data is assessed, with one or more control processes being actuated based thereon.
(36) A method of automatically monitoring and controlling water, the method comprising: providing at least one rule in a memory of a water monitoring and control system, the rule comprising at least one parameter and when triggered, causing a signal to activate one or more of a notification, a water flow device and an automated sampling device of the water monitoring and control system; determining via a processor of the system whether the rule has been triggered; if the processor determines that the rule has been triggered, determining via the processor whether the at least one parameter meets a predetermined threshold for a predetermined deactivation duration and if yes, determining via the processor that a parameter threshold condition is not met and causing the rule to no longer be triggered, and if no, determining via the processor that the parameter threshold condition is met; and if the processor determines that the rule has not been triggered, determining via the processor whether the at least one parameter exceeds the predetermined threshold for a predetermined activation duration and if so, determining via the processor that the parameter threshold condition is met and causing the rule to be triggered, and if no, determining via the processor that the parameter threshold condition is not met.
(37) A method of automatically monitoring and controlling water of claim 36 or any clause herein, wherein when the parameter threshold condition is not met, the rule ceases to be triggered.
(38) A method of automatically monitoring and controlling water of claim 36 or any clause herein, wherein when the parameter threshold condition is met, the rule continues to be triggered.
(39) A real-time water monitoring and control system according to any clause herein, wherein the system includes a rule engine and a user interface for at least one of creating, adjusting and customizing one or more rules for the rule engine thereof, wherein the user interface is configured to a plurality of conditions to be combined in creating a rule of the rule engine, including one or more of rainfall conditions, one or more system state conditions, one or more water quality and flow conditions and one or more activation and deactivation conditions, all of which are required to be satisfied in order to trigger the rule and actions associated therewith.
(40) A real-time water monitoring and control system according to any clause herein, including a rule engine and a rule creation and adjustment interface thereof, the rule creation and adjustment interface enabling one or more of: i) customization of pH thresholds of the water with customization of one or more amounts of time past which pH levels of the water outside of said pH thresholds are unacceptable, triggering the system to recirculate rather than discharge the water; ii) customization of turbidity thresholds of the water with customization of one or more amounts of time past which turbidity levels of the water outside of said turbidity thresholds are unacceptable, triggering the system to recirculate rather than discharge the water; iii) customization of the rainfall amount and rolling time period thresholds which comprise said rainfall condition; iv) customization of the time period required for a rule to be activated; and v) customization of the time period required for a rule to be deactivated.
(41) A real-time water monitoring and control system according to any clause herein, including a rule engine and a user interface thereof, with the user interface enabling: customizable notifications of escalating urgency comprising actions of the system activated in response to whether and to the extent to which the water complies with the water quality thresholds; customization of the frequency within which said notifications are sent; and customization of persons to receive a given said notification depending on the importance thereof.
(42) A real-time water monitoring and control system according to any clause herein, including a dashboard interface which displays via visual indicia raw water quality and rainfall data in real-time, including visual indicia of rainfall amount totals as a function of time, visual indicia of pH levels of the water as a function of time, visual indicia of turbidity levels of the water as a function of time, and visual indicia of temperature levels of the water as a function of time.
(43) A real-time water monitoring and control system according to any clause herein, wherein the system includes a remote server comprising a rule engine with the water quality thresholds and real time data collection of water quality and flow of the effluent stored and logged on memory thereof.
(44) A computer program product comprising a medium carrying computer readable instructions which, when executed by a processor, cause the processor to execute a process according to any clause herein.
(45) A server comprising a computer program product according to clause 44 or any clause herein, the server being configured to deliver the computer readable instructions to a recipient computer by a way of a communication medium.
(46) Apparatus including any new and inventive feature, combination of features, or sub-combination of features as described herein.
(47) Methods including any new and inventive steps, acts, combination of steps and/or acts or sub-combination of steps and/or acts as described herein.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

What is claimed is:

1. A real-time water monitoring and control system comprising:
a plurality of sensors configured to measure properties of the water in real-time; and
a processor in communication with the sensors and rainfall data in real-time, the processor being configured to cause the water to discharge based on whether the water is below a first water quality threshold and the processor being configured to cause the water to discharge based on whether the water is below a second water quality threshold where a rainfall condition has been met.

2. A real-time water monitoring and control system of claim 1, wherein the processor is configured to determine, in parallel or sequentially in any order, i) one or more of water quality and flow of the water based on the plurality of sensors; and ii) whether the rainfall condition has been met.

3. A real-time water monitoring and control system of claim 1, including i) one or more of a rainfall gauge and a weather application programming interface (API) integration to obtain rainfall data in real-time based on a given location and time; ii) water quality said sensors via which the processor determines water quality; and iii) one or more flow said sensors via which the processor determines water flow.

4. A real-time water monitoring and control system of claim 1, wherein the second water quality threshold is higher than the first water quality threshold.

5. A real-time water monitoring and control system of claim 1, wherein the processor recirculates the water through a water treatment system where the rainfall condition has not been met and the water quality of the water as determined by the sensors exceeds the first water quality threshold and wherein the processor recirculates the water through a water treatment system where the rainfall condition has been met and the water quality of the water as determined by the sensors exceeds the second water quality threshold.

6. A real-time water monitoring and control system according to claim 1, wherein the system includes a rule engine and a user interface for at least one of creating, adjusting and customizing one or more rules for the rule engine thereof, wherein the user interface is configured to a plurality of conditions to be combined in creating a rule of the rule engine, including one or more of rainfall conditions, one or more system state conditions, one or more water quality and flow conditions and one or more activation and deactivation conditions, all of which are required to be satisfied in order to trigger the rule and actions associated therewith.

7. A real-time water monitoring and control system according to claim 1, including a rule engine and a rule creation and adjustment interface thereof, the rule creation and adjustment interface enabling one or more of: i) customization of pH thresholds of the water with customization of one or more amounts of time past which pH levels of the water outside of said pH thresholds are unacceptable, triggering the system to recirculate rather than discharge the water; ii) customization of turbidity thresholds of the water with customization of one or more amounts of time past which turbidity levels of the water outside of said turbidity thresholds are unacceptable, triggering the system to recirculate rather than discharge the water; iii) customization of the rainfall amount and rolling time period thresholds which comprise said rainfall condition; iv) customization of the time period required for a rule to be activated; and v) customization of the time period required for a rule to be deactivated.

8. A real-time water monitoring and control system according to claim 1, including a rule engine and a user interface thereof, with the user interface enabling: customizable notifications of escalating urgency comprising actions of the system activated in response to whether and to the extent to which the water complies with the water quality thresholds; customization of the frequency within which said notifications are sent; and customization of persons to receive a given said notification depending on the importance thereof.

9. A real-time water monitoring and control system according to claim 1, including a dashboard interface which displays via visual indicia raw water quality and rainfall data in real-time, including visual indicia of rainfall amount totals as a function of time, visual indicia of PH levels of the water as a function time, visual indicia of turbidity levels of the water as a function of time, and visual indicia of temperature levels of the water as a function of time.

10. A real-time water monitoring and control system according to claim 1, wherein the system includes a remote server comprising a rule engine with the water quality thresholds and real time data collection of water quality and flow of the effluent stored and logged on memory thereof.

11. A real-time water monitoring and control process comprising:
obtaining rainfall data in real-time via one or more of a rain gauge and a weather application programming interface (API) and measuring water quality, flow and system data in real-time via one or more sensors;
determining in real time via a processor whether the rainfall data satisfies a set of one or more predetermined rainfall conditions;
in parallel or sequentially, in any order, determining in real time whether one or more of the water quality, flow and system data satisfies a set of one or more of predetermined conditions, and if so, sending a signal to trigger or activate one or more of a notification, a water flow device and an automated sampling device; and
sending a signal to deactivate all triggered actions when any said predetermined condition is no longer satisfied.

12. A real-time water monitoring and control process according to claim 11, wherein for the one or more predetermined rainfall conditions, the process includes determining one or more of i) whether the rainfall amount calculated based on a rolling window of a predetermined duration with an end point being the current moment exceeds a predetermined threshold and ii) whether the rainfall amount exceeded the predetermined threshold at an earlier point in time but within another predetermined duration with the end point being the current moment.

13. A real-time water monitoring and control process according to claim 11, wherein for the one or more predetermined conditions, the process includes determining whether said one or more of the water quality, flow and system data satisfy a predetermined comparison operator when evaluated against a predetermined threshold thereof for a predetermined duration thereof with the end point being the current moment.

14. A real-time water monitoring and control process according to claim 11, wherein the real-time water monitoring and control system is configured to automatically and continually query third party rainfall data API based on location and time parameters and wherein the water flow device comprises one or more of a valve and a pump.

15. A real-time water monitoring and control process according to claim 11, whereby if the rainfall data exceeds the set of one or more predetermined rainfall conditions, the processor is configured to determine whether said one or more of the water quality, flow and system data exceeds a first said set of predetermined conditions and if so, the processor is configured to send a first signal to activate one or more of a first said notification, the water flow device and the automated sampling device, and whereby if the rainfall data is below the set of one or more predetermined rainfall conditions, the processor is configured to determine whether said one or more of the water quality, flow and system data exceeds a second said set of predetermined conditions and if so, the processor is configured to send a second signal to activate one or more of a second said notification, the water flow device and the automated sampling device.

16. A real-time water monitoring and control process according to claim 15, wherein the processor is configured to determine whether the rainfall data exceeds the set of one or more predetermined rainfall conditions for a first predetermined period of time and if so, the processor is configured to determine whether said one or more of the water quality, flow and system data exceeds the first said set of predetermined conditions for one or more predetermined periods of time and if so, the processor is configured to send the first signal to activate one or more of the first said notification, the water flow device and the automated sampling device and wherein if the rainfall data is below the set of one or more predetermined rainfall conditions for the first predetermined period of time, the processor is configured to determine whether said one or more of the water quality, flow and system data exceeds a second set of one or more predetermined conditions for one or more predetermined periods of time and if so, the processor is configured to send the second signal to activate one or more of the second said notification, said water flow device and said automated sampling device.

17. A real-time water monitoring and control system comprising the one or more rain gauge and the weather API integration, the one or more sensors and the processor according to a real-time water monitoring and control process of claim 11.

18. A computer program product comprising a medium carrying computer readable instructions which, when executed by a processor, cause the processor to execute a process according to claim 15.

19. A server comprising a computer program product according to claim 18, the server being configured to deliver the computer readable instructions to a recipient computer by a way of a communication medium.

20. A method of automatically monitoring and controlling water, the method comprising:
obtaining rainfall data in real-time via one or more of a rainfall gauge and a weather application programming interface (API) integration configured to obtain rainfall data in real-time based on a given location and time;
determining via a processor whether the rainfall data for a first predetermined amount of time exceeds a predetermined threshold and if so recording that a new significant rainfall event has occurred and determining that a rainfall condition has been met,
if the processor determines that the rainfall data for the first predetermined amount of time does not exceed the predetermined threshold, determining via the processor whether a previous significant rainfall event was recorded and if no, determining that the rainfall condition has not been meet;
if the processor determines that the rainfall data for the first predetermined amount of time does not exceed the predetermined threshold and determines that the previous significant rainfall event was recorded, determining via the processor whether the previous significant rainfall event was recorded at a time equal to or less than a second predetermined amount of time and if so, determining that the rainfall condition has been met;
if the processor determines that the rainfall data for the first predetermined amount of time does not exceed the predetermined threshold, determines that the previous significant rainfall event was recorded, and determines that the previous significant rainfall event was recorded at a time greater than the second predetermined amount of time, determining via the processor that the rainfall condition has not been met; and
determining whether water is to be discharged based on different water quality criteria depending on whether the rainfall condition has been met.

21. A real-time water monitoring and control process comprising:
continuously monitoring water at a construction site having wastewater;
automatically sending a first signal to activate one or more control processes upon determining that one or more of water quality, flow and system data exceeds one or more predetermined value thresholds for greater than a first predetermined amount of time; and
automatically sending a second signal to deactivate the one or more control processes upon determining the one or more of water quality, flow and system data is below the one or more predetermined value thresholds for greater than a second predetermined amount of time.

22. A real-time water monitoring and control process according to claim 21, including one or more of: determining the one or more of water quality, flow and system data via one or more water quality and flow sensors; and continuously monitoring rainfall data at or adjacent the construction site and using said rainfall data in real-time to determine the one or more predetermined value thresholds against which the water quality, flow and system data is assessed, with one or more control processes being actuated based thereon.
